(19) United States
(12) Patent Application Publication
(10) Pub. No.: US 2026/0351426 A1
(43) Pub. Date: Nov. 5, 2026

(54) VISION BASED DETECTION OF BREASTFEEDING RELATED CONDITIONS

(71) Applicant: The Regents of the University of California, Oakland, CA (US)

(72) Inventors: Edward Jay Wang, La Jolla, CA (US); Jessica de Souza, La Jolla, CA (US); Kristina Chamberlain, La Jolla, CA (US)

(21) Appl. No.: 19/485,629

(22) PCT Filed: May 23, 2024
(86) PCT No.: PCT/US2024/030884

§ 371 Date: Nov. 18, 2025

Related U.S. Application Data
(60) Provisional application No. 63/503,936, filed on May 23, 2023.

(51) Int. Cl. G16H 80/00; G06N 3/0464; G16H 30/20; G16H 40/67; G16H 50/20

(52) U.S. Cl. CPC G16H 80/00; G06N 3/0464; G16H 30/20; G16H 40/67; G16H 50/20

(57) ABSTRACT

An artificial intelligent (AI) mediated patient management system is described in this disclosure. This patient management system includes a patient interface configured to collect breastfeeding-related information from a patient, the breastfeeding-related information being stored in images and/or videos, and a semi-independent system connected to the patient interface and configured to propose actionable feedback to the patient. The semi-independent system includes a data pre-processing module connected with the patient interface and configured to resize and normalize the images and/or videos forwarded by the patient interface, and a convolutional neural network (CNN) engine having a final layer and a softmax activation function that classify the processed images into one or more possible issues of a plurality of possible issues, comprising normal, mastitis, and nipple damage. The patient management system also includes a patient triage module and a provider interface connected to the patient triage module and a lactation consultant.

### CROSS-REFERENCE TO RELATED APPLICATION(S)

The present international application claims priority to U.S. Provisional Patent Application No. 63/503,936, filed on May 23, 2023. The entire contents of the above noted provisional application are incorporated by reference as part of the disclosure of this document.

### TECHNICAL FIELD

The present disclosure generally relates to vision based detection of breastfeeding related conditions, and more particularly relates to detecting nipple damage and mastitis in lactation consulting videos using deep learning neural network models.

### BACKGROUND

Breastfeeding is a critical period in the postpartum phase that offers numerous health benefits for both the mother and the infant. It is a natural process that, while beneficial, can present a range of challenges that may discourage mothers from continuing to breastfeed. These challenges can include various treatable medical conditions and a lack of adequate support. The cessation of breastfeeding due to such factors is a significant concern, as it deprives the infant of the optimal nutrition and immune protection that breast milk provides.

Common issues faced by breastfeeding mothers include nipple damage and mastitis, affecting approximately 80% and 20% of breastfeeding mothers, respectively. Nipple damage can lead to pain and discomfort, making the breastfeeding experience distressing and leading some mothers to discontinue the practice prematurely. Moreover, mastitis as an inflammation of the breast tissue can result in infection. Symptoms may include breast pain, swelling, warmth, redness, and flu-like symptoms. Like nipple damage, mastitis can be a deterrent to continued breastfeeding if not promptly and effectively treated.

In response to these challenges, many mothers seek assistance from lactation consultants (LCs), who are healthcare professionals specializing in the science of breastfeeding. LCs are trained to address a wide array of concerns related to breastfeeding, including but not limited to, issue with milk supply, breast and nipple problems, and strategies for milk management upon a mother's return to work. They also provide prenatal education to expectant mothers, preparing them for the breastfeeding journey ahead.

### SUMMARY

An artificial intelligent (AI) mediated patient management system is described in this disclosure. This patient management system includes a patient interface configured to collect breastfeeding-related information from a patient, the breastfeeding-related information being stored in images and/or videos, and a semi-independent system connected to the patient interface and configured to propose actionable feedback to the patient. The semi-independent system includes a data pre-processing module connected with the patient interface and configured to resize and normalize the images and/or videos forwarded by the patient interface, wherein the data pre-processing module generates processed images having a fixed size and format, and a convolutional neural network (CNN) engine having a final layer and a softmax activation function that classify the processed images into one or more possible issues of a plurality of possible issues, comprising normal, mastitis, and nipple damage. The patient management system also includes a patient triage module that includes a data base containing the one or more possible issues; and a provider interface connected to the patient triage module and a lactation consultant, the provider interface configured to enable the lactation consultant to communicate with the patient to diagnose one of the plurality of possible issues. In the present technology, the patient interface is directly connected to the provider interface and the semi-independent system is not directly connected to the patient trigae system. In addition, the classification of the processed images is transferred from the semi-independent system to the provider interface through the patient interface.

In another general aspect, a method for breastfeeding related issue management is described in this disclosure. This method includes inputting, from a patient interface, a patient's breastfeeding-related information to data pre-processing module of a semi-independent system, the breastfeeding-related information being stored in images and/or videos, and resizing and normalizing the images and/or videos, by the data pre-processing module, to generate processed images having a fixed size and format. Additionally, this method includes integrating the processed images to a CNN engine of the semi-independent system for inference and classifying, by the CNN engine, the processed images into one or more possible issues of a plurality of possible issues comprising normal, mastitis, and nipple damages. Further, this method includes outputting the one or more possible issues to the patient through the patient interface and transferring classification result from the CNN engine to a provider interface through the patient interface.

### BRIEF DESCRIPTION OF THE DRAWINGS

FIG. 1 illustrates a perinatal patient management system in accordance with various embodiments of the present technology.

FIG. 2 illustrates an AI-mediated patient management pipeline in accordance with various embodiments of the present technology.

FIG. 3 shows a CNN engine adopted in the AI-mediated patient management pipeline in accordance with various embodiments of the present technology.

FIGS. 4A to 4C show line drawing images of a healthy lactating breast, a breast with visible mastitis infection, and a breast with a damaged nipple, respectively in accordance with various embodiments of the present technology.

FIG. 5 illustrates an image processing pipeline in processing images and video data for breastfeeding issue evaluation, in accordance with various embodiments of the present technology.

FIG. 6 shows performance of five CNN engines on the 7-class dataset: (A) Resnet50, (B) VGG16, (C) EfficientNetV2, (D) InceptionV3, and (E) DenseNet169, in accordance with various embodiments of the present technology.

FIG. 7 shows algorithm performance on 7-class dataset after data augmentation, corresponding to the five CNN engines illustrated in FIGS. 6A to 6E and in accordance with various embodiments of the present technology.

FIG. 8 shows an aggregated confusion matrix for the Resnet50 CNN engine for the augmented dataset with example results from the augmented dataset that were correctly and incorrectly classified across all folders, in accordance with various embodiments of the present technology.

FIG. 9 shows model performance on the binary dataset without and with augmentation in accordance with various embodiments of the present technology.

FIG. 10 shows an aggregated confusion matrix for the Resnet50 CNN engine for the augmented dataset with example results from the augmented dataset that were correctly and incorrectly classified across all folders, in accordance with various embodiments of the present technology.

FIGS. 11A to 11G show example line drawing images from the testing set of the CNN deep learning engine, in accordance with various embodiments of the present technology.

FIGS. 12A to 12F show example line drawing images leading to correct prediction from several classes of model misclassification, in accordance with various embodiments of the present technology.

FIGS. 13A, 13B, and 13C show example incorrectly classified line drawing images due to common visual features in accordance with various embodiments of the present technology.

FIG. 14A to 14C show incorrectly classified line drawing images due to dataset variety limitations in accordance with various embodiments of the present technology. FIG. 14A shows a healthy breast classified as mastitis. FIG. 14B shows a breast with mastitis classified as healthy. FIG. 14C shows a nipple damage image classified as mastitis.

FIG. 15 is a flow chart illustrating a method for breastfeeding related issue management in accordance with various embodiments of the present technology.

### DETAILED DESCRIPTION

Breastfeeding offers numerous advantages for both the infant and the mother, including reduced incidence of gastrointestinal infections in the child, accelerated postpartum weight loss for the mother, and extended periods of amenorrhea. These health benefits are among the primary reasons why physicians globally advocate for a minimum of six months of breastfeeding. In light of these advantages, along with heightened awareness and advocacy efforts, breastfeeding initiation rates have seen a significant uptick in the United States. In 2014, a notable 79% of newborns were introduced to breastfeeding.

Despite the strong evidence supporting breastfeeding and its rising initiation rates, many families encounter challenges in maintaining it. Data reveals a stark decline in breastfeeding duration, with rates dropping to below 41% at three months and dwindling further to 19% for exclusive breastfeeding by six months. Parents who choose to breastfeed grapple with various obstacles, such as insufficient milk production, fatigue associated with breastfeeding, medical contraindications, difficulties with latching or other feeding techniques, pain during breastfeeding, and a lack of supportive networks.

The expertise LCs is invaluable in ensuring a smooth and painless transition into breastfeeding, thereby increasing the likelihood of mothers breastfeeding for the recommended duration of six months or longer. However, the availability of LCs is limited, creating a significant service gap. The dramatic mismatch between the number of mothers requiring assistance and the number of available LCs has led to a reliance on telehealth systems. Through these systems, mothers can reach out to LCs by sharing images and descriptions of their breastfeeding issues to receive timely advice and support. Despite the benefits of telehealth, the limited number of LCs remains a bottleneck in providing adequate breastfeeding support. Currently, the availability of International Board Certified Lactation Consultants (IBCLCs) around the globe is limited. In 2021, there were in total 3.6 million births in the U.S. and only 18.5 k LCs with IBCLC certification, being a rate of 194 babies per LC a year. Meanwhile, in lower-resource countries, such as Brazil, in the same year, there were a total of 2.6M births for a total of 154 certified LCs, a rate of 16,883 babies per LC a year. The high demand for LC professionals is notable, especially in Low and Middle income Countries (LMICs) such as Brazil, where parents may have more difficulties finding LCs for breastfeeding guidance. Therefore, it is crucial to develop better tools for LCs that allow them to provide more immersive and supportive experiences for the parents. The demand for LCs keeps increasing due to the health benefits of breastfeeding, preventative care to mitigate issues that may lead to breastfeeding cessation and increase of parents willing to breastfeed long-term.

In addition, access to LCs is limited to geographic availability, leaving less urbanized areas with decreased breastfeeding assistance. Another issue is professional availability itself, since most LCs combine their practice with being midwife nurses, splitting their time between prenatal visits, attending births, lactation consultations and managing their patients, often overloading these groups and being correlated with professional exhaustion and emotional stress. Most LCs are independent service providers, and uninvolved with clinics that contain complex patient systems, especially outside the United States. The lack of availability to patient management and triage systems for LCs add complexity to their time management and organization of their own schedule. This shortage underscores the need for innovative solutions that can enhance the accessibility and effectiveness of breastfeeding support, thereby addressing the gap in lactation care and promoting the health and well-being of mothers and their infants.

To resolve the above described limitations and challenges, the present technology introduces an enhancement to telehealth services for postpartum care by incorporating a deep-learning algorithm designed to identify breastfeeding-related conditions. In particular, the present technology outlines an automated telehealth pipeline tailored for postpartum care. Further, the present technology evaluates the potential for a deep learning model to detect nipple damage and mastitis in lactation consulting videos. Moreover, a thorough error analysis has been conducted to pinpoint image or video features that could potentially mislead illness detection models.

The present disclosure presents that a transfer-learned classification model achieves above 0.9 Area under ROC Curve (AUC) in detecting breast-related conditions and 90% accuracy in the multi-class classification task of detecting specific conditions. Consequently, the automatic vision-based illness detection systems of breastfeeding-related conditions presented in the present disclosure uncover an opportunity for augmenting telehealth pipelines to improve post-partum care for new mothers. Specifically, the present disclosure sets forth the following contributions to the technology: 1) the present technology introduces a computer vision-based method for triaging breast conditions, designed for integration within telehealth pipelines; 2) the present technology entails an evaluation of a computer vision model using a curated online image dataset of breastfeeding conditions, comprising images of healthy subjects (N=153), subjects with nipple damage (N=126), and subjects with mastitis (N=93); and 3) the present technology also includes an evaluation of both the performance of the model and the data quality derived from videos captured using naturalistic smartphone settings.

In addition, the present disclosure presents a best CNN engine achieves an average area under the AUC of 0.93 for all conditions after data augmentation for multi-class classification. For binary classification, the present technology has achieved with the best model an average AUC of 0.96 for all conditions after data processing.

### Supporting LCs Through Tele-Lactation Services

The present disclosure discloses a computer vision-based triaging of breast conditions that can be integrated into a telehealth pipeline for postpartum lactation care. The assessment of breast condition is important as pain and discomfort experienced during breastfeeding is a major barrier faced by parents who want to continue to provide their child with breastfeeding. It is reported that 80% of mothers are estimated to suffer from nipple pain and fissures, and 20% are estimated to experience mastitis. This telehealth pipeline for postpartum lactation care incorporates automatic detection of visually discernible breastfeeding-related conditions such as nipple cracks, fissures related to poor latching and positioning, and risk of mastitis spectrum issues. The automatic detection uses a deep-learning computer vision model, which can benefit triaging of lactation patients for a more efficient and focused patient response based on levels of severity.

As described in the present disclosure, the performance of the proposed deep-learning model to distinguish between healthy and unhealthy (for mastitis and nipple damage conditions) has been evaluated through an image and video evaluation. In particular, the image evaluation uses 74 images, and the video evaluation uses 15 videos, with both evaluations having the data split into the three conditions. In the image evaluation, the deep-learning model achieves AUC above 0.9 for the three conditions and can detect healthy breast images with 83% accuracy, and unhealthy breast images with 85% accuracy for mastitis and nipple damage with 93% accuracy. In the video evaluation, the deep-learning model was able to correctly classify 9 videos with correctness above 99% (5 healthy videos, 2 mastitis videos, and 2 nipple damage videos) and 3 videos with inferior correctness between 31% and 53% (1 mastitis video and 2 nipple damage videos), having 3 videos with 0% correctness (2 mastitis videos, and 1 nipple damage video). The video evaluation of the current technology, curated from recordings taken under highly naturalistic lighting and environmental conditions, offers a thorough examination of how image capture can impact performance. This analysis provides valuable insights for future user interface designs and the guidance necessary for the effective use of such systems.

### The Postpartum Telehealth Pipeline

The subsequent subsections of the present disclosure detail three principal areas critical to the enhancement of a postpartum telehealth pipeline, including the current state of the art of hybrid lactation consultations, issues associated with breastfeeding and their visuals, and current technological advances in breast-related image recognition.

### Lactating Care Pipeline: In-Person, Remote, and Hybrid

Mothers who have access to prenatal care are introduced to the possibility of breastfeeding by their healthcare provider. They are provided with printed or online resources to inform them about breastfeeding prior to giving birth. Right after birth, depending on the type of delivery (cesarean vs. vaginal), the mother may or may not be able to breastfeed right after the baby's birth. Most hospitals around the globe follow the UNICEF/WHO baby-friendly initiative, which includes guidelines for successful breastfeeding, envisioning the mother's and baby's health, and providing resources and alternatives for mothers who may not follow this path. Once the mother leaves the hospital, it is the family's responsibility to seek support in case they encounter any difficulties with breastfeeding. This support can be sought from friends, relatives, healthcare facilities, and LCs.

LCs typically provide in-person postpartum consultations due to the hands-on nature of their work. They often begin assisting their patients from the first minute of the baby's life until the mother no longer needs breastfeeding support. LCs conduct visual and physical evaluations of both the mother and baby. This evaluation includes assessing the baby's internal mouth structure, the anatomy of the breast and nipple, milk supply, and assisting the mother in ensuring proper attachment or repositioning of the baby to prevent nipple fissures. Additionally, LCs may introduce laser therapy as a treatment option for damaged nipples resulting from the misuse of breast pumps or issues with baby attachment and use dolls and breast plushies with internal anatomy to educate the mother in how milk production happens and how positioning affects the milk extraction. Overall, the immersive and hands-on approach of LCs is crucial for providing personalized and effective lactation support to mothers and infants.

With the high adoption of smartphones communication apps worldwide, several public health facilities in urban areas such as family clinics started to provide patient services such as appointment scheduling, health orientations, and vaccine campaign notifications in developing countries. The communication apps became popular communication tools between patients and healthcare providers, including breastfeeding education and family support with neonates.

When LCs transitioned to virtual consultations due to the COVID-19 pandemic, they incorporated into their workplace apps that the population is already familiar with, such as Whatsapp, Instagram, and Facebook. These communication apps were already consolidated by 2020. However, LCs needed these tools while remodeling their approach to accommodate the mother's needs with the same quality but without the resources from in-person consultations. Lactation care and other modalities that require physical evaluation, like physical therapy, needed to reinvent themselves to achieve patient satisfaction and solve cases when the scenario switched from in-person to remote. As such, LCs used communication and social media apps to provide remote consultations and educate parents using features these apps provide and having larger visibility.

Prior research has identified several challenges encountered in remote lactation care, including a limited field of view of the patient (mother and baby) during video consultations, difficulties in communicating instructions to the patient, challenges faced by mothers in demonstrating their breastfeeding issues, the absence of standardized guidelines, and various technical difficulties. In contrast, remote lactation care brings benefits and advantages, such as helping combat the mother's feeling of isolation, allowing the LC to provide faster feedback and reassurance to their patients during virtual consultations, and enabling LC to focus on effective communication, which benefits the patient's independent learning since it requires higher engagement between patient and LC during consultations. This independent learning positively impacts mothers' intentions in exclusive breastfeeding up to six months and reduces the risk of breastfeeding cessation at three months by 25%.

Previous research showed fully remote consultations as a model that works for cases where the patient and provider cannot meet in person due to geographic distance, transportation issues, or patient disease. Additionally, LCs conduct remote consultations from their workplace (personal office, clinics or hospitals) for situations where LCs who are also midwives are on call to attend births and have time between their duties. The profile of patients in which LCs provide consultation includes patients who seek them pre-birth, post-birth, and emergency cases where the mother is already facing breastfeeding challenges. Depending on the nature of the meeting, the patient may have different needs, which can be met through in-person or remote visits. Follow-up visits and cases that require LC's fast response has been preferred by LCs to be conducted remotely. In summary, remote care is seen as a beneficial increment to in-person care, not a replacement, especially to provide a secure environment for mothers to seek guidance, reassurance, and confidence and lack access to such an environment at home.

The nature of LC's work makes their time availability limited, especially for the ones who are also midwives. LCs claim in previous research to have high demand for patients and respond to several patient remote messages a day, in which some concerns have higher priority than others. Scrolling through all the patients and deciding which patient to address first takes time and inconvenience for the mothers who need priority. The present technology uncovers a computer vision-based triaging of breast conditions that can incorporate a telehealth pipeline and help LCs better understand the patients that need just-in-time responses in remote settings.

Issues Associated with Breastfeeding

One of the reasons correlated with breastfeeding cessation is the pain associated with breastfeeding which can be caused by issues such as poor attachment of the baby onto the breast, physical conditions on mother or baby, misuse of breast pumps, oversupply of breast milk, and even environmental conditions. These issues appear in the first few days after birth, and when not treated can last up to a few weeks, jeopardizing the continuity of breastfeeding above 6 months of life. Some of the conditions can be fully mitigated when the mother receives orientation and education in the topic, such as poor attachment and misuse of breast pumps, while other conditions can be alleviated and managed for a better experience for the mother, like physical conditions including nipple physiology, baby tongue-tie and jaw clenching, and dealing with excessive milk supply.

In the present disclosure, two groups of problems that lead to pain in breastfeeding have been investigated. The first problem is the mastitis spectrum and resolved by the present technology. Mastitis is a painful condition that includes symptoms such as redness in the breast, and may involve flu-like symptoms, hardened skin surface in the location of the milk blockage and even blood in the milk. About 20% of mothers who breastfeed may face issues within the mastitis spectrum disorder within their time breastfeeding, which can include breast engorgement, phlegmon, inflammatory and bacterial mastitis, and breast abscess. When the mastitis evolves to a breast abscess, it is required medications and even medical procedures to drain the inflammation from the breast.

Another problem resolved by the present technology is nipple damage caused by improper latching and positioning from the infant, excessive pressure from breast pumping devices, infant tongue-tie or palate abnormality, abnormal rhythm of the infant to express milk, and even infant biting or jaw clenching. Considering the cause of nipple damage, 80% of mothers are expected to face some level of nipple issues during breastfeeding, which, if not treated, may cause an average of 35% of these mothers to cease breastfeeding before 1 month. Nipple damage is painful and may be visible or invisible. When visible, it can present features at the skin surface such as fissures, cracks, pus, blood, scarring or crusting. In the present disclosure, nipple blisters has been categorized into the nipple damage group, which may be associated with mastitis in its beginning. However, due to the nature and location of the condition nipple blisters was grouped as nipple damage. Additionally, some skin dermatoses such as thrush, herpes, eczema, and psoriasis are also included in the group of nipple damage due to its occurrence more presence in the nipple.

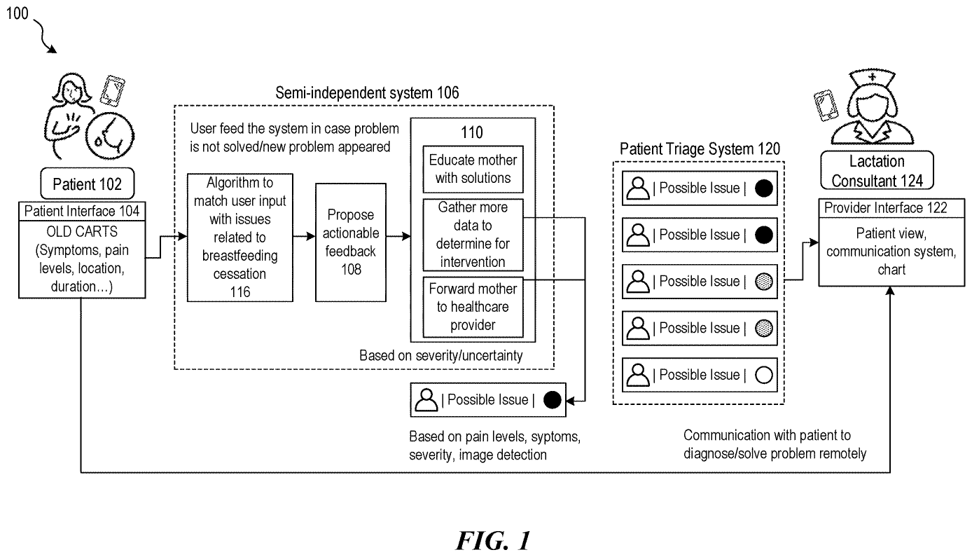

FIG. 1 discloses a perinatal patient management system 100 that is configured to assist LCs 124 in providing postpartum care to patients 102 via a telehealth platform in accordance with various embodiments of the present technology. The perinatal patient management system 100 comprises a patient interface 104, a semi-independent system 106, a propose actionable feedback 108, a patient triage system 120, and a provider interface 122. In this example, the patient interface 104 is a device or an application that enables the patient 102 to communicate with the semi-independent system 106 and the LC 124, and to provide information about their breastfeeding-related conditions, such as symptoms, pain levels, location, duration, and images. The semi-independent system 106 includes a deep-learning algorithm (e.g., the algorithm 116) that analyzes the information provided by the patient 102 and matches it with possible issues related to breastfeeding cessation, such as mastitis, nipple damage, engorgement, or low milk supply.

In this example, the semi-independent system 106 may not diagnose the patient 102, but rather proposes actionable feedback 108 to the patient 102 based on the information and the possible issues. The propose actional feedback 108 is a communication system that delivers the feedback from the semi-independent system 106 to the patient 102, such as suggestions, tips, or referrals. In particular, the semi-independent system 106 may offer additional information 110 to the patient 102, such as educating mother with solutions, gathering more data to determine need for intervention, and forwarding mother to healthcare provider.

As shown in FIG. 1, the patient triage system 120 includes a database that contains one or more possible issues to the patient. It also provide source information regarding one or more medical issues to the LC 124. The patient triage system 120 may also prioritize specific possible issues based on the severity, urgency, or complexity of their condition. The provider interface 122 is a device or an application that enables the LC 124 to access the information stored in the patient triage system 120 and the feedback of the patient 102 through the communicate with the patient 102 via the patient interface 104. In this example, the provider interface 122 can be directly connected to the patient interface 104 for information exchange and intercommunication. Specifically, the semi-independent system 106 may not be directly connected to the patient triage system 120 nor the provider interface 122. In this example, the LC 124 is a health care professional who specializes in breastfeeding support and education, and who can diagnose, treat, or refer the patient 102 as needed.

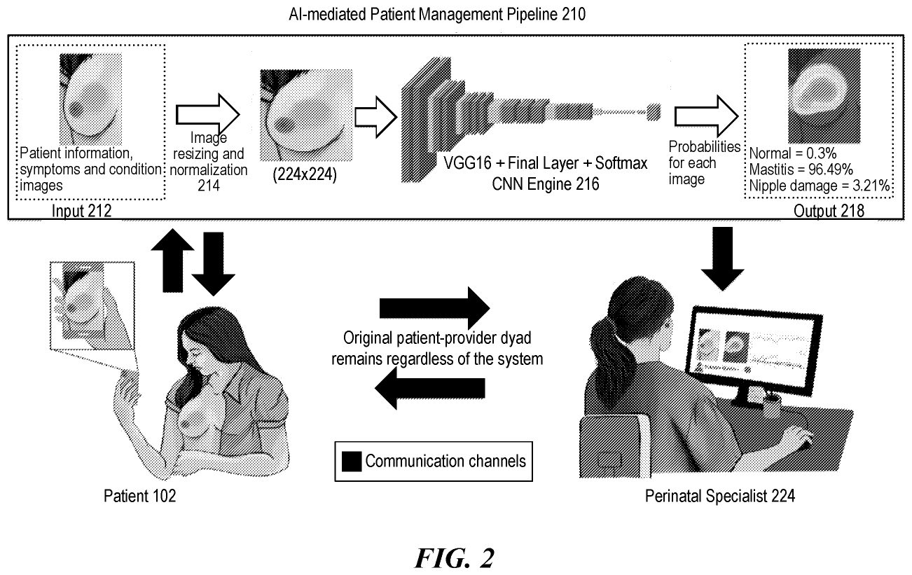

FIG. 2 illustrates an AI-mediated patient management pipeline 210 in accordance with various embodiments of the present technology. The AI-mediated patient management pipeline 210 can be facilitated in the semi-independent system 106 of FIG. 1 as an algorithm to match user input with issues related to breastfeeding cessation. In this example, an exemplary embodiment of an AI-mediated patient management pipeline 210 is provided for diagnosing and treating breast conditions in lactating women. As shown, the pipeline 210 receives as input 212 of patient information, symptoms, and condition images from a patient 102 and provides output 218 to a perinatal specialist 224. The input 212 is processed by an image resizing and normalization module 214 that transforms the condition images to a standard size and format (e.g., 224×224 pixels) suitable for the subsequent deep learning module 216 to process. In this example, the normalization of images involves modifying the pixel values in images to have a specific statistical distribution. For example, the image resizing and normalization module 214 can scale the image pixel values to a specified range or adjusting the pixel values so that the mean of the pixel values and the standard deviation become specific values. The normalization ensures that the input images have a consistent scale, which is crucial for comparison and processing in CNN algorithms that are sensitive to the scale of input data. In some other examples, the image resizing and normalization module may process the image of input 212 to other sizes such as 128×128 pixels, 256×256 pixels, 512×512 pixels, and 1024×1024 pixels. In some other examples, the image resizing and normalization module may process the images having non-RGB channels at the input 212, such as CMYK channels, HSV channels, infrared channels, and multi-spectral images.

As shown in FIG. 2, the deep learning module 216 comprises a VGG16 CNN engine with a final layer and a softmax activation function that classifies the condition images into one of three categories: normal, mastitis, or nipple damage. The deep learning module 216 outputs 218 as the probabilities of each category for the input 212, which can be used to inform the diagnosis and treatment decisions of the patient 102 or the perinatal specialist 224. The pipeline 210 maintains the original patient-provider dyad regardless of the system and enables communication channels between the patient 102 and the perinatal specialist 224. In this example, the classification results output from the CNN engine 216 can be forwarded to the perinatal specialist 224 in assisting LC's diagnosis. For example, the classification results generated by the VGG16 engine can be transferred from the semi-independent system 106, through the patient interface 104, to the provider interface 122, as illustrated in FIG. 1.

In some other examples, the AI-mediated patient management pipeline 210 may incorporate another deep learning CNN engines, such as a LeNet-5 model, an AlexNet model, a VGGNet model, a GoogLeNet model, and/or a ResNet model. In some other examples, the AI-mediated patient management pipeline 210 can be configured to classify the condition images into one of many categories including breast cancer, fibrocystic breasts, mastitis, breast cysts, papilloma, gynecomastia, breast abscess, breast calcification, and/or breast atrophy.

Moreover, the present disclosure includes investigation of the effectiveness of five additional distinct CNN engines in detecting healthy lactating breasts and six breastfeeding-related issues by only using RGB images. The goal is to assess the applicability of this algorithm as an auxiliary resource for LCs to identify painful breast conditions quickly, better manage their patients through triage, respond promptly to patient needs, and enhance the overall experience and care for breastfeeding mothers.

Specifically, the present disclosure evaluates the potential for the corresponding five classification models to detect breastfeeding-related conditions using 1,078 breast and nipple images gathered from online and physical educational resources. For example, CNN engines such as Resnet50, VGG16, InceptionV3, EfficientNetV2, and DenseNet169 have been adopted to classify the images across seven classes: healthy, abscess, mastitis, nipple blebs, dermatosis, engorgement, and nipple damage by improper feeding or misuse of breast pumps. The present disclosure also includes evaluation of the CNN engines' ability to identify between healthy and unhealthy images.

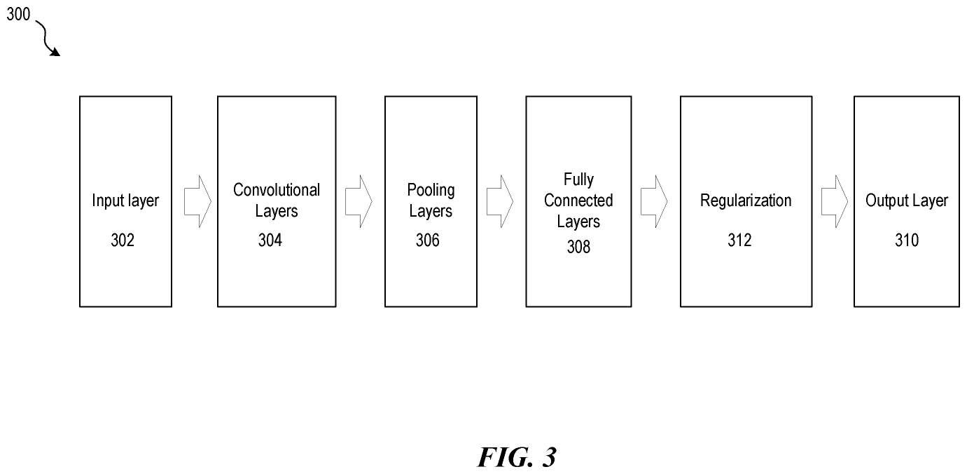

FIG. 3 shows a CNN deep learning engine 300 that can be adopted in the AI-mediated patient management pipeline 210 described on FIG. 2. The CNN deep learning engine 300 can be structured in a series of layers that each perform different transformations on the input data to extract features, and eventually lead to a classification output. For example, the CNN deep learning engine 300 includes an input layer 302, which can take an image of fixed size 224×224 pixels with three channels (RGB). In addition, the CNN deep learning engine 300 can include a plurality of convolutional layers 304. These convolutional layers 304 use small filters (e.g., 3×3) to capture local features in the input images. Each of the convolutional layers 304 can be followed by a ReLU activation function which introduces non-linearity to the model, helping it to learn more complex patterns.

In this example, several max pooling layers 306 can be interspersed between the convolutional layers. These max pooling layers 306 can perform down-sampling by taking the maximum value in a particular window (e.g., 2×2) and sliding this window across the previous layer's output. After several convolutional layers 304 and max pooling layers 306, the high-level processing in the network is done via fully connected layers 308. In the CNN deep learning engine 300, the output layer 310 is a softmax layer which outputs the probabilities of the instance belonging to each of up to 1000 classes. In this example, the CNN deep learning engine 300 may also include a regularization layer 312, which is placed after the fully connected layers 308 to prevent overfitting. Here, each convolutional layer 304 and fully connected layer 308 have learnable parameters (e.g., weights and biases), which are adjusted during the training phase using backpropagation.

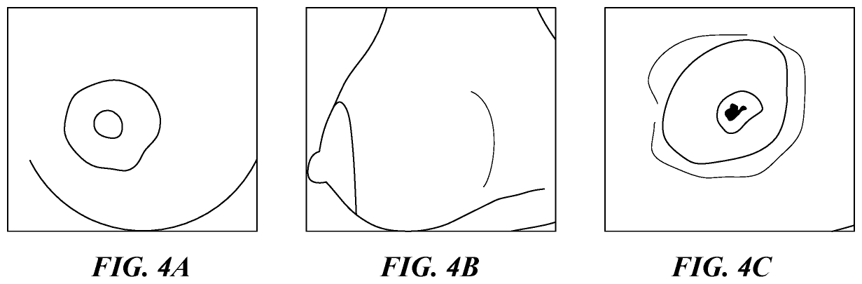

FIG. 4 presents the visual differences between a healthy lactating breast compared to a breast with mastitis and a damaged nipple. In particular, FIGS. 4A to 4C show line drawing images of a healthy lactating breast, a breast with visible mastitis infection, and a breast with a damaged nipple, respectively in accordance with various embodiments of the present technology. In this example, FIG. 4 present sample line drawing images used as dataset containing a visual description of three categories of breastfeeding-related disorders that will be used in the image classification of the present technology. For example, FIG. 4A shows a normal breast with no visual conditions, which belongs to the healthy group. FIG. 4B shows a breast with visible mastitis infection containing large red spots on the breast. In addition, FIG. 4C shows a damaged nipple in the scarring phase caused by poor latching of the baby onto the breast. It can be noticed that the healthy breast does not contain any signs of redness in the breast and no signs of laceration in the nipple. The breast with mastitis visually shows large spots of redness throughout the breast and signs of swelling. The breast with nipple damage shows parts of scarring, tissue exposure and blood.

### Breast-Related Image Recognition

It was demonstrated that the effectiveness of deep learning for the evaluation of breast images for applications such as malign and benign breast tumors for both lactating and non-lactating women, helping improve the accuracy of breast ultrasound and mammogram exams. Those works involved using medical imaging previously taken in medical facilities to improve the evaluation of breast-related illnesses and allow better accuracy in the diagnosis for medical personnel. Furthermore, research on in-hospital postpartum stays has demonstrated predicting exclusive breastfeeding outcomes and helping support professional decision-making.

In the realm of breastfeeding disorders, there exists a notable gap in research concerning the application of deep learning algorithms for the analysis of authentic images to identify abnormalities, including mastitis, nipple fissures, and abscesses. For example, there are reported agriculture work involving early prediction of mastitis, in which the risk of mastitis is constantly assessed to prevent a reduction in milk production from animals and affecting dairy industry. This highlights a critical need for research aimed at adapting these technologies to detect and prevent breastfeeding disorders in humans. Such research is particularly vital in environments with limited access to medical professionals and LCs. By enabling early detection and prevention, these advancements can reduce the incidence of breastfeeding cessation, enhance maternal-infant bonding, and improve the overall health and well-being of mothers and infants.

### Deep Learning Model and Methods of Evaluation

The present technology utilizes a breast image dataset, which is a compilation of physical and digital images specifically curated for training and validating a deep learning model's ability to distinguish between healthy and unhealthy lactating breasts. The dataset contains images labeled according to their respective conditions, e.g., healthy lactating breast, nipple injury due to various causes, and breast with mastitis. Preparing this dataset presents certain challenges due to the need for diversity in skin color, breast and nipple size, and image capture angle and lighting conditions. In the present disclosure, a dataset comprising 153 images of healthy breasts, 126 images showcasing nipple damage, and 74 images indicative of the mastitis spectrum has been adopted. The images were sourced from various platforms, including breastfeeding-related books, articles, online blogs for mothers and physicians, YouTube from educative organizations, and social media platforms. Each image in the dataset is labeled according to its corresponding condition. For example, ‘healthy’ for healthy breasts that may or may not show signs of breast fullness. In addition, ‘nipple damage’ for breasts and nipples exhibiting signs of trauma caused by abnormal latch or pump, thrush, blisters, or dermatoses in the nipple. Further, ‘mastitis’ for breasts showing signs of inflammatory or bacterial mastitis, early signs of breast abscess and phlegmon.

In another example, the present technology uses a breast image dataset (see Table 1), a compilation of physical and digital images specifically curated to train and validate our deep learning model's ability to distinguish between healthy and unhealthy lactating breasts. This dataset includes images categorized according to their respective conditions: healthy lactating breast, nipple injuries due to various causes, nipple blebs due to plugged ducts, breast or nipple with signs of dermatoses, and breasts in the conditions of engorgement, mastitis, and abscess.

Here, in order to be included in the dataset, images must meet the following criteria: (1) the image must be in RGB format, either as PNG or JPEG; (2) it must visually have at least one of the seven conditions; (3) the breast and/or nipple should be visible; (4) the image should be hosted in a trustworthy source (from medical professionals such as physicians, midwife nurses, and IBCLCs), in which the image must have a word or description identifying its condition among the seven classes to be included as its label; (5) the visual condition present in the image and the label provided describing the condition should match. Images were excluded from the dataset if (1) the breast or nipple were from non-lactating female patients, (2) if the condition described on the label and the visual features of the image did not match, (3) the breast or nipple was not visible in the image, and (4) if the image did not have any label describing it. In addition, the present technology can invite a board-certified nurse practitioner (CNM, ARNP, IBCLC) with more than 15 years of experience to perform a final review of the dataset to ensure that images and labels had no discrepancies.

In this example, images were collected from diverse sources such as breastfeeding-related books, articles, online blogs for mothers and physicians, YouTube from educative organizations, and social media platforms (e.g., Instagram, Facebook, Twitter) from certified healthcare providers who would have educative resources for mothers. To ensure diversity in geographical and racial representation, image searches using multiple languages (e.g., English, Portuguese, Spanish, French, Chinese) and search engines adjusted for other countries were also conducted.

TABLE 1
Dataset description.

| Dataset size | 393.7 MB, each image: [min: 0.015, average: 0.360, max: 3.575] MB | | | | | | | |
|---|---|---|---|---|---|---|---|---|
| Dimensions | Width [min: 68, average: 606, max: 2448] | | | | | | | |
| | Height [min: 68, average: 607, max: 2448] | | | | | | | |
| Number of | 1078 | | | | | | | |
| images | | | | | | | | |
| Number of | 7 | | | | | | | |
| classes | | | | | | | | |
| Number of | 586 | | | | | | | |
| unique subjects | | | | | | | | |
| Number of | Abscess: 115 | | | | | | | |
| Images per | Dermatoses: 123 | | | | | | | |
| class | Engorgement: 63 | | | | | | | |
| | Mastitis: 180 | | | | | | | |
| | Nipple bleb: 82 | | | | | | | |
| | Nipple damage: 197 | | | | | | | |
| | Healthy: 318 | | | | | | | |
| Visual features | Abscess: swelling and redness, area with palpable fluid collection, pus | | | | | | | |
| per class | Dermatoses: rash, discoloration, flaky skin, uneven skin tone, crusting, redness | | | | | | | |
| | Engorgement: swelling, redness, skin stretched and shiny, enlarged nipple | | | | | | | |
| | Mastitis: red patches on breast and/or nipple, swelling, pus, or blood discharge | | | | | | | |
| | Nipple bleb: small white or yellow bumps on nipple or areola, similar to a blister | | | | | | | |
| | Nipple damage: nipple swelling, redness, peeling/flaking skin, bleeding, shape | | | | | | | |
| | differences | | | | | | | |
| | Healthy: regular breast and nipple color, may have visible veins | | | | | | | |
| Number of | Physical: 178 (books, magazines, articles) | | | | | | | |
| images per | Physician Websites: 366 | | | | | | | |
| source | YouTube: 65 (educational channels on women's health) | | | | | | | |
| | Other: 469 (received by LCs, IBCLC's Instagram, Google Images, and Flickr, support | | | | | | | |
| | groups mediated by LCs on social media and other educational websites) | | | | | | | |
| | Class Name | FST I | FST II | FSTI III | FST IV | FST V | FST VI | N/A |
| Number of | Abscess | 28 | 35 | 20 | 8 | 14 | 8 | 2 |
| images per skin | Dermatoses | 17 | 37 | 48 | 13 | 3 | 3 | 2 |
| tone per class | Engorgement | 4 | 6 | 18 | 30 | 4 | 0 | 1 |
| (Fitzpatrick skin | Mastitis | 44 | 69 | 51 | 11 | 1 | 4 | 0 |
| type (FST) [57]) | Nipple bleb | 9 | 16 | 18 | 8 | 6 | 3 | 22 |
| | Nipple damage | 40 | 59 | 22 | 15 | 11 | 5 | 45 |
| | Healthy | 61 | 90 | 92 | 21 | 28 | 21 | 5 |
| | Total per FST | 203 | 312 | 269 | 106 | 67 | 44 | 77 |

In this example, images can be collected from a diverse group of female patients with several skin colors, breast and nipple sizes, with unstandardized image sizes, orientations, backgrounds, and light sources. In total, the dataset consists of 1078 images, with 318 images of healthy breasts, 115 images of breast abscesses, 123 images of dermatoses, 63 images of breast engorgement, 180 images of mastitis, 82 images of nipple blebs, and 197 images of nipple damage. As a general rule for images collection, a healthy lactating breast presented a uniform color, was free of redness, and had no signs of discharge. Nipples are expected to exhibit a variety of shapes, including flat, protruded, or inverted, and varying in sizes. In engorgement, images showed breast and nipple swelling, skin stretched and shiny, and some light redness due to high milk production. For nipple blebs or nipple damage, signs of laceration, blood, blisters, and redness are expected. Mastitis showed swelling, redness, and discharge of pus or blood in the nipple. Abscess shares similarities with mastitis but involves worsened redness and pus in the infected region and may display signs of rupture. Finally, dermatosis images contained signs of skin rash, breast or nipple uneven skin tone, and crusting.

In addition to the image dataset, the present technology includes a video evaluation assessing the feasibility of real-time breastfeeding condition detection to emulate realistic image quality captured by individual users in uncontrolled lighting and environments. These videos provide context based on which the deep learning model of the present technology can perform in realistic conditions and elucidate potential limitations. Furthermore, these videos provide additional qualitative information about how patients naturally capture videos of their breasts, providing insight into future user interface implementation and user education needed to guide users to capture high-quality, machine-readable images. In this disclosure, the test evaluates 15 video footage of breasts with various lighting, angles and skin tones. The average time for each video is 4.93 seconds (standard deviation of 2.02 seconds), and the average number of frames per video is 190 (standard deviation of 109.78 frames). These frames were extracted from the videos and were labeled according to their corresponding condition.

In preparation for utilization as inputs in the deep learning model, the images undergo a preprocessing sequence to optimize the deep learning model performance and enhance accuracy. Initially, the images are cropped to maintain uniform dimensions of 224×224 pixels, encompassing exclusively the breast and nipple tissue. Specifically, the image resizing and normalization module 214 resizes input images to a square geometry. This selective cropping concentrates the model's analytical focus on the areas of greatest relevance. Subsequently, the images are subjected to normalization by implementing a mean and standard deviation normalization technique. This step is designed to refine the model's functionality by equalizing the pixel intensity distribution throughout the dataset, thereby facilitating more consistent and reliable analysis.

With the present technology, the performance of five CNN engines commonly used in computer-vision problems including VGG16, Resnet50, InceptionV3, EfficientNetV2, and DenseNet169 have been examined. All models were built with the PyTorch library for image classification, in which the models had all layers frozen except for the last layer, which was replaced with a fully connected layer adapted to the number of classes-two for binary classification and seven for the multi-class task. In this example, all CNN engines were trained for 100 epochs using the AdamW optimizer with a learning rate of 3e⁻⁴, weight decay of 0.1, and batch size of 20. Here, 100 epochs were chosen since it was a converging point where the accuracy no longer increased or decreased. For the loss functions, Binary Cross-Entropy with Logits Loss was applied for binary classification tasks, and Cross-Entropy Loss was used for multi-class tasks. Both were fine-tuned with class weights to strategically adjust for class imbalances by proportionally penalizing misclassifications in less represented classes. These models were evaluated using stratified K-Fold cross-validation with 10 folds. To ensure the robustness of the cross-validation process, any learned parameters were reset by initializing the models from scratch at the beginning of each fold. Instead of using the entire image dataset to train the model, feature extraction was conducted to optimize the training process (detailed in the Feature Extraction section). The performance of the five CNN engines were also measured across the same data and keep the hyperparameters the same: learning rate, weight decay, batch size, and number of epochs.

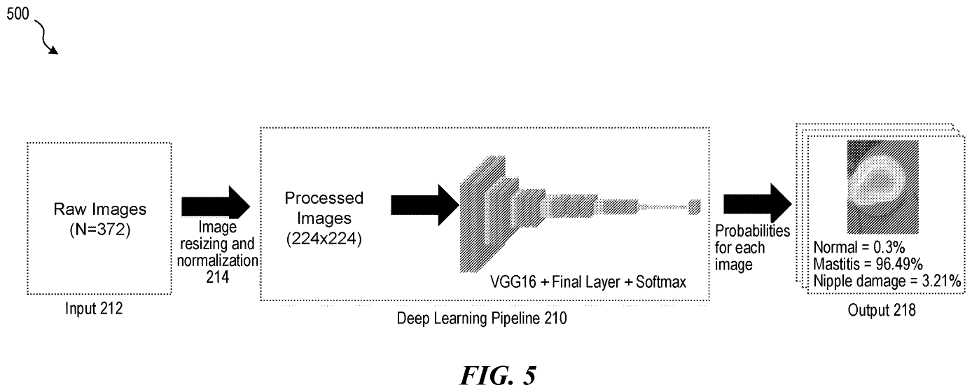

FIG. 5 illustrates an image processing pipeline 500 in processing images and video data for breastfeeding issue evaluation, in accordance with various embodiments of the present technology. As shown, the image processing pipeline 500 is configured to accept breast images as input 212. Within this pipeline 500, a deep learning pipeline 210 processes each image by first resizing it to a predetermined scale and subsequently normalizing the images to standardize pixel intensity values across the dataset. This pre-processing 214 readies the images for effective analysis by the model. Following the preprocessing phase, the deep learning model/pipeline 210 employs a classification algorithm to assign a probability to each image, indicating its likelihood of belonging to one of three predefined breastfeeding issue categories. The category corresponding to the highest probability is then selected as the output 218 to represent the most likely condition of the image. This method ensures accurate and efficient image classification within the image processing pipeline 500.

In this example, the present technology employs a CNN engine VGG16, with the pre-trained model built using a PyTorch library, for image classification. Here, the VGG16 model consists of 16 layers in total: 13 convolutional layers and 3 fully connected layers. Except for the last layer, all layers of the VGG16 model are frozen so that their weights will not change during training. The last layer is replaced with a fully connected layer comprising 3 outputs, enabling the application of transfer learning to enhance the deep learning model's performance with the custom breast and nipple ailment dataset. In this example, the VGG16 model is fine-tuned over 100 epochs using the Adam optimizer with a learning rate of 3×10⁻⁴ and a cross-entropy loss function. The dataset is randomly divided between training and testing sets, e.g., with 80% of the images allocated for training and 20% for testing. No validation set is included in this example because no hyperparameters, other than the learning rate, are tuned for this study. Here, the deep learning model's performance is evaluated based on several metrics, including True Positive Rate (TPR, or recall), True Negative Rate (TNR, or specificity), Positive Prediction Value (PPV, or precision), Accuracy, False Positive Rate (FPR), False Negative Rate (FNR), and the area under the Receiver Operating Characteristic (ROC) curve (AUC).

In some other examples, various CNN engines can be adopted in the present technology for the breast image classification. Each of the CNN engines may include one or more of an input layer, convolutional layers, an activation function layer, pooling layers, fully connected layers, and an output layer. For example, the present technology may adopt a LeNet-5 CNN engine for the imaging pipeline shown in FIG. 5. The LeNet-5 model may consist of convolutional layers, subsampling layers, and fully connected layers. In another example, the present technology may adopt an AlexNet model, which features convolutional layers, max pooling, dropout layers, and fully connected layers, and employs ReLU activation functions to enhance non-linearity. In another example, the present technology may adopt a VGGNet model for the image processing. This VGGNet model can be good for simplicity, using only 3×3 convolutional filters stacked on top of each other in increasing depth to improve system depth and network performance. In some other examples, the present technology may adopt a GoogleNet model for the imaging pipeline. The GoogLeNet model allows the neural network to choose from filters of various sizes in the same layer, and significantly increases the depth and width of the neural network while keeping computational requirements in check. In some other example, the present technology may adopt a ResNet model, by introducing “skip connections” or “shortcut connections” that allow the network to skip one or more layers, for the imaging pipeline.

Before inputting raw images into the deep-learning CNN engines, as shown in FIG. 5, the raw images were manually cropped to ensure images were deidentified and had no irrelevant content, such as unrelated body areas, clothes, jewelry, identifiable tattoos, or backgrounds, enhancing the model's accuracy and performance. In particularly, the images were cropped in a 1:1 ratio to prevent image flattening or warping during resizing and loss of important features. Most images have breast and nipple tissue concentrated in the center of the image, thereby focusing the model's evaluation on the most relevant areas. This image preprocessing guidelines aims to objectively show the area of interest for optimized detection and reduce risks of poorly triaged images.

After cropping the images in a 1:1 ratio and before entering the deep learning pipeline 210, some standard transformations can be applied in the data, starting with image resizing. In this disclosure, the dataset has been trained, validated, and tested using five different CNN deep learning models. Four of the chosen models (VGG16, Resnet50, EfficientNetV2, and DenseNet169) specify the input images to be resized to 224×224 pixels, and the model InceptionV3 model requires input images to be resized to 299×299 pixels. Therefore, the image resizing was proceeded according to each model's requirements. The last transformation step incorporates normalization of the images, a procedure where the pixel intensity values are standardized across the dataset. To help the models generalize better for our dataset, the mean and standard deviation of all images in the dataset has been calculated and used in the normalization process instead of using the ImageNet dataset pretrained parameters.

The curation of the dataset revealed a limitation in the number of images per class due to the challenges associated with collecting images and the variability in clinical features. To address these issues and to prevent overfitting, data augmentation techniques were employed in the present technology. These methods served to artificially increase the dataset by creating realistic transformations of the existing images. Six specific data augmentations, previously utilized in skin lesion datasets, were applied: center zoom, random rotation, brightness adjustment, shear, and both vertical and horizontal flips. Initially, the dataset comprised 1078 images, which expanded to 6478 following augmentation. The comprehensive figures detailing the sample counts before and after augmentation are presented in Table 2.

In the present disclosure, the dataset underwent evaluation both prior to and subsequent to the data augmentation. Originally, 1000 images were designated for training and validation, divided using stratified k-fold cross-validation with 10 folds. This division allowed for 90% of the data to be used for training and 10% for validation within each fold, ensuring that the proportion of images in each class was maintained across both training and validation sets. This stratification guaranteed that each fold accurately represented the dataset as a whole. A set of 78 images was kept separate from these folds, reserved solely for final testing to gauge the deep learning model's accuracy on new data. Following the augmentation, the dataset was increased to 6000 images for training and validation purposes. The test set was also enlarged to 468 images to align with the augmented training data, while still providing a robust evaluation of the model on unseen examples.

In some examples, feature extraction was performed using five models pre-trained on the ImageNet dataset. This method effectively reduced the number of computational resources required for processing the dataset. It transformed images into numerical features while preserving pertinent information. The models were configured to operate in evaluation mode, where feature maps are derived from the final convolutional layers. Subsequently, these maps underwent adaptive pooling and were flattened into one-dimensional arrays. The resulting extracted features were stored and utilized as input for the model classifiers.

TABLE 2
Detailed number of samples on the dataset

| | | | | Train | Test |
|---|---|---|---|---|---|
| | | Train | Test | Samples | Samples |
| Dataset | Classes | Samples | Samples | (Augmented) | (Augmented) |
| 7-class | Abscess | 108 | 7 | 648 | 42 |
| | Dermatoses | 115 | 8 | 690 | 48 |
| | Engorgement | 55 | 8 | 330 | 48 |
| | Mastitis | 171 | 9 | 1026 | 54 |
| | Nipple bleb | 75 | 7 | 450 | 42 |
| | Nipple | 188 | 9 | 1128 | 54 |
| | damage | 288 | 30 | 1728 | 180 |
| | Healthy | | | | |
| Binary | Unhealthy* | 657 | 40 | 3942 | 240 |
| | Healthy* | 343 | 38 | 2058 | 228 |

*Unhealthy class combines the classes abscess, dermatoses, mastitis, nipple bleb and nipple damage, while the healthy class combines healthy and engorgement, all from the 7-class dataset.

In the present disclosure, a total of five CNN engines were trained on the dataset. The study proposed four tasks to evaluate the CNN engines across different datasets: (1) multi-class not augmented, (2) multi-class augmented, (3) binary not augmented, and (4) binary augmented. As described in Table 2, an additional two evaluations were performed considering a binary model to assess the CNN engines' ability to differentiate between healthy and unhealthy images. The unhealthy class consolidates five conditions: abscess, dermatoses, mastitis, nipple bleb, and nipple damage. Conversely, the healthy class combines the original healthy and engorgement conditions. In the binary evaluation, engorgement images were included in the healthy condition because engorgement is not inherently indicative of disease and often resolves without medical intervention. Moreover, engorgement shares visual characteristics with healthy breast conditions, which might not be distinguishable at an early, non-problematic stage. All CNN engines underwent k-fold cross-validation, with performance metrics collected from each fold and their average computed. The performance of the CNN engines for both the multi-class and binary datasets was assessed using the same metrics: accuracy, precision, recall, F1-score, and the Receiver Operating Characteristic Area Under the Curve (ROC-AUC).

### Video Detection Evaluation

The present disclosure also includes a comprehensive validation of the deep learning model by utilizing videos from the three classes instead of static images. These videos represent short scenes displaying the breast area, showcasing various motions, angles and distances from the subject. These videos simulate scenarios such as performing a smartphone-based breast scan or engaging in a video call where the LC assesses the patient during a virtual consultation. This analysis also helps understand whether the accuracy improves by combining several frames from the same individual.

In this example, a small dataset containing 15 videos have been created. These videos were collected from educational online channels in the context of each of the three classes. Specifically, there are five videos for each of the categories. The average duration per video is 4.53 seconds (standard deviation of 1.99 s), with an average number of frames per video of 165.76 (standard deviation of 80.36). The evaluation pipeline has each video with its frames extracted, with the same preprocessing pipeline as the previous evaluation described earlier in this disclosure. After that, the model prediction scores across all frames were computed, each containing the probability of belonging to each of the three classes. In this example, the prediction score is a continuous number between 0 and 1, where the video is classified as a condition if the score exceeds 0.5.

### Image Quality, Image Interpretations, and Video Interpretations

In this disclosure, the evaluation of five CNN engines focuses on their capability to differentiate between healthy conditions and six other breastfeeding-related issues. Table 3 displays the aggregated evaluation metrics for each CNN, organized by test accuracy. Metrics such as precision, recall, F1-score, and the area under the Receiver Operating Characteristic (ROC-AUC) curve are provided as weighted averages. This approach compensates for class imbalances within the datasets, ensuring proportional contribution of each class to the final metric based on its prevalence. For the cross-validation process, distinct test sets were utilized for each fold to assess the CNNs, with the reported metrics representing the mean of these evaluations.

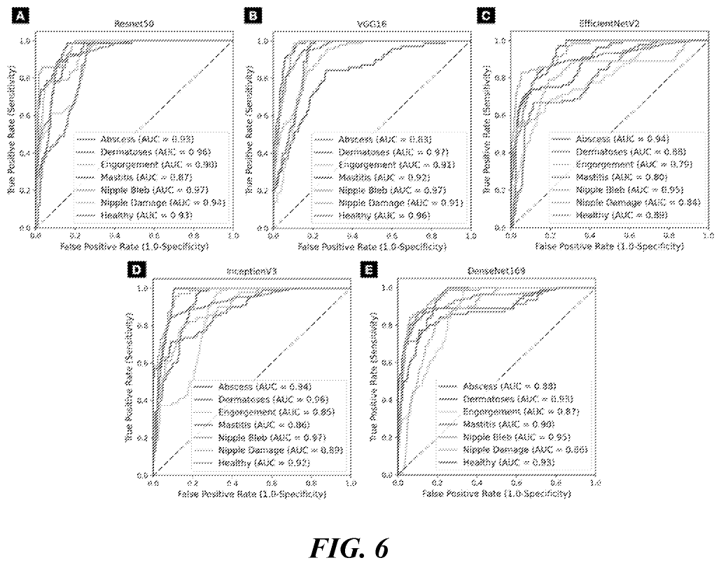

The Resnet 50 emerged as the top-performing model, demonstrating the highest test accuracy, closely followed by the VGG16 and EfficientNetV2, which showed a slight difference in performance. When applying a similar weighted average approach in a one-vs-rest configuration, the CNN engines attained an overall ROC-AUC of 0.934 for VGG16, 0.929 for Resnet50, 0.912 for InceptionV3, 0.908 for Densenet169, and 0.872 for EfficientNetV2. FIG. 6 illustrates the detailed ROC-AUC for each class across the different models.

TABLE 3
Average evaluation metrics for the trained models on the
not augmented dataset (sorted based on performance).

| | | Training | Validation | Test Set Metrics | | | |
|---|---|---|---|---|---|---|---|
| Dataset | Model | Accuracy | Accuracy | Accuracy | Precision | Recall | F1-score |
| 7-class | Resnet50 | 0.907 | 0.737 | 0.608 | 0.675 | 0.623 | 0.637 |
| | VGG16 | 0.818 | 0.678 | 0.604 | 0.674 | 0.589 | 0.600 |
| | EfficientNetV2 | 0.779 | 0.626 | 0.604 | 0.658 | 0.582 | 0.593 |
| | InceptionV3 | 0.903 | 0.727 | 0.574 | 0.680 | 0.607 | 0.622 |
| | DenseNet169 | 0.932 | 0.771 | 0.507 | 0.659 | 0.596 | 0.572 |

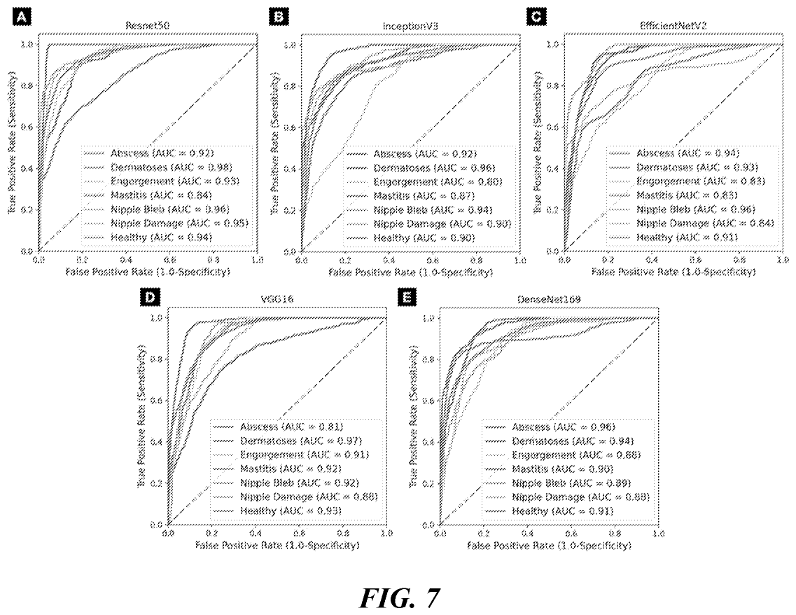

The application of data augmentation to the multi-class model involved providing a broader array of images to enhance the model's ability to generalize from the training data without altering the original class distribution. The outcomes, as presented in Table 4 and FIG. 7, indicate improved metrics for most of the CNN engines following data augmentation. Notably, Resnet50 emerged as the top-performing model. The models recorded a Receiver Operating Characteristic-Area Under Curve (ROC-AUC) score of 0.934 for Resnet50, 0.912 for VGG16, 0.909 for Densenet169, 0.898 for InceptionV3, and 0.893 for EfficientNetV2.

TABLE 4
Average evaluation metrics for the trained models on
the augmented dataset (sorted based on performance).

| | | Training | Validation | Test Set Metrics | | | |
|---|---|---|---|---|---|---|---|
| Dataset | Model | Accuracy | Accuracy | Accuracy | Precision | Recall | F1-score |
| 7-class | Resnet50 | 0.953 | 0.907 | 0.672 | 0.717 | 0.715 | 0.713 |
| augmented | InceptionV3 | 0.920 | 0.844 | 0.617 | 0.692 | 0.637 | 0.649 |
| | EfficientNetV2 | 0.803 | 0.808 | 0.602 | 0.650 | 0.586 | 0.599 |
| | VGG16 | 0.755 | 0.801 | 0.585 | 0.644 | 0.561 | 0.563 |
| | DenseNet169 | 0.954 | 0.889 | 0.506 | 0.639 | 0.611 | 0.563 |

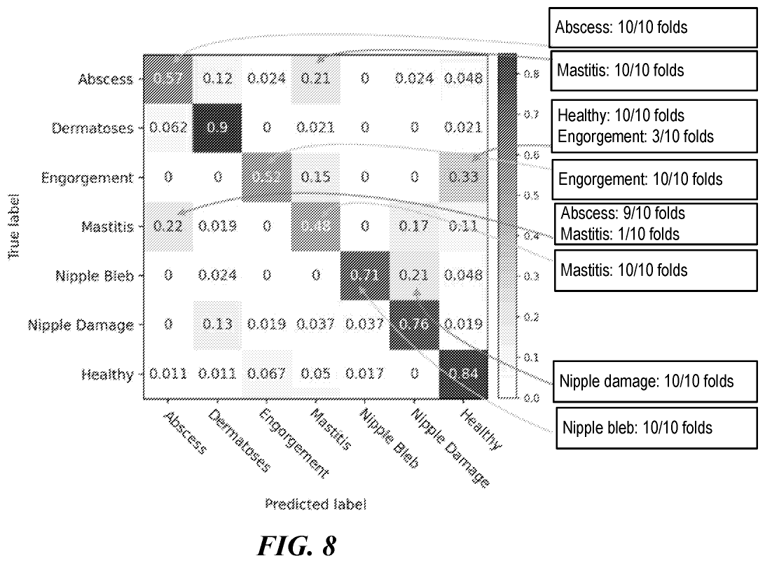

Upon examining the performance of the optimal model, specifically the Resnet50 with the augmented dataset, a closer look at the metrics per class of this CNN engines is warranted. Table 5 displays the results for 10-fold cross-validation, showcasing an overall consistent performance throughout the iterations. FIG. 8 illustrates the aggregated confusion matrix for the Resnet50 model. This matrix consolidates the predictions from all ten iterations when applied to the augmented dataset. The aggregation process involved taking the median predicted class for each instance over the multiple folds. This method synthesizes a singular prediction that represents the consensus of the model's behavior across the test set.

TABLE 5
Results of 10-fold Cross-Validation for the
augmented dataset on Resnet50

| 10-Fold | | | | |
|---|---|---|---|---|
| Iterations | Accuracy | Precision | Recall | F1-score |
| Iteration 1 | 0.699 | 0.705 | 0.699 | 0.699 |
| Iteration 2 | 0.714 | 0.715 | 0.714 | 0.712 |
| Iteration 3 | 0.709 | 0.713 | 0.709 | 0.709 |
| Iteration 4 | 0.729 | 0.730 | 0.729 | 0.727 |
| Iteration 5 | 0.718 | 0.719 | 0.718 | 0.716 |
| Iteration 6 | 0.733 | 0.734 | 0.733 | 0.730 |
| Iteration 7 | 0.720 | 0.722 | 0.720 | 0.718 |
| Iteration 8 | 0.707 | 0.711 | 0.707 | 0.706 |
| Iteration 9 | 0.707 | 0.707 | 0.707 | 0.705 |
| Iteration 10 | 0.720 | 0.715 | 0.720 | 0.713 |

Out of the 468 images in the testing set, the present technology accurately classified 341 images. Specifically, the breakdown of correctly classified images by category is as follows: abscess (N=24, Acc=57%), dermatoses (N=43, Acc=90%), engorgement (N=25, Acc=52%), mastitis (N=26, Acc=48%), nipple bleb (N=30, Acc=71%), nipple damage (N=41, Acc=76%), and healthy (N=152, Acc=84%). Misclassifications occurred among visually similar conditions and those that can precede each other. Table 6 presents the performance of the selected model for each class within the augmented test set. The model struggled with distinguishing abscesses, which were falsely identified as dermatoses and mastitis in 12% and 21% of the images, respectively. Breast engorgement was incorrectly classified as mastitis and healthy breasts in 15% and 33% of the cases, respectively. Mastitis was mistaken for abscess in 22% of cases, nipple damage in 17%, and healthy breasts in 11%. Additionally, 21% of nipple bleb images were incorrectly classified as nipple damage.

TABLE 6
Summary of the detection results per class: accuracy,
precision, recall, F1-score and support (number of samples
per class) using the Resnet50 architecture.

| | | | | F1- | |
|---|---|---|---|---|---|
| Class | Accuracy | Precision | Recall | score | Support |
| Abscess | 0.571 | 0.585 | 0.571 | 0.578 | 42 |
| Dermatoses | 0.895 | 0.729 | 0.895 | 0.804 | 48 |
| Engorgement | 0.520 | 0.641 | 0.520 | 0.575 | 48 |
| Mastitis | 0.481 | 0.481 | 0.481 | 0.481 | 54 |
| Nipple bleb | 0.714 | 0.857 | 0.714 | 0.779 | 42 |
| Nipple damage | 0.759 | 0.683 | 0.759 | 0.719 | 54 |
| Healthy | 0.844 | 0.844 | 0.844 | 0.844 | 180 |

### Binary Image Detection Evaluation

In this disclosure, the dataset was streamlined from seven categories to two primary ones: healthy and unhealthy, in order to enhance the precision of clinical predictions and minimize the likelihood of erroneous outcomes. The unhealthy category encompasses five specific conditions: abscess, dermatoses, mastitis, nipple bleb, and nipple damage. The healthy category is expanded to include the original healthy conditions along with engorgement. Due to the visual similarities between engorgement and other healthy breast conditions, distinguishing engorgement proved challenging for multi-class models. It was observed that 33% of engorgement images were misclassified as healthy. Table 7 displays the consolidated evaluation metrics for five different models, organized by test accuracy.

TABLE 7
Average evaluation metrics for the trained models on the not
augmented binary dataset (sorted based on test accuracy).

| | | Training | Validation | Test Set Metrics | | | |
|---|---|---|---|---|---|---|---|
| Dataset | Model | Accuracy | Accuracy | Accuracy | Precision | Recall | F1-score |
| Binary | VGG16 | 0.901 | 0.877 | 0.876 | 0.990 | 0.760 | 0.859 |
| | Resnet50 | 0.923 | 0.872 | 0.841 | 0.954 | 0.715 | 0.817 |
| | InceptionV3 | 0.906 | 0.845 | 0.838 | 0.963 | 0.702 | 0.812 |
| | EfficientNetV2 | 0.866 | 0.831 | 0.811 | 0.991 | 0.629 | 0.769 |
| | DenseNet169 | 0.935 | 0.880 | 0.761 | 0.990 | 0.529 | 0.688 |

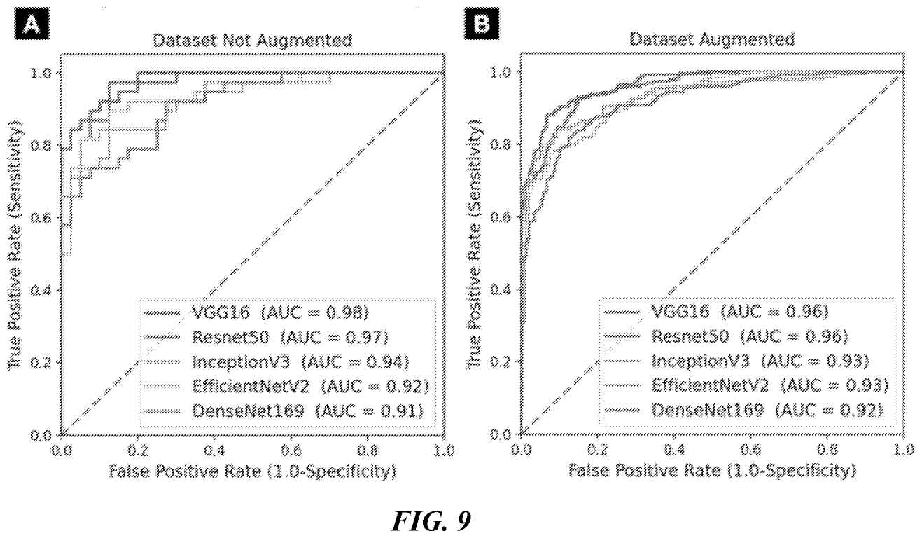

The accuracy metric is calculated as a balanced score to compensate for any class imbalances, ensuring equitable contribution from each category to the overall metric. Metrics such as precision, recall, and F1-score are provided for the positive category, with a clear indication of the positive class label. In the cross-validation process, a distinct test set was utilized for each fold to assess the model's performance, and the metrics presented are the mean of these evaluations. Among the models tested, VGG16 emerged as the most accurate, outperforming others like Resnet50 and InceptionV3. The models registered an overall receiver operating characteristic curve area under the curve (ROC-AUC) of 0.977 for VGG16, 0.966 for Resnet50, 0.935 for InceptionV3, 0.921 for EfficientNetV2, and 0.910 for Densenet169. FIGS. 9A and 9B detail the ROC-AUC for datasets without augmentation and with augmentation, respectively.

In the present technology, applying data augmentation to the binary model involved providing a wider variety of images to enhance the model's ability to generalize from the training data without altering the original class distribution. The results, as presented in Table 8, indicate improvements across the CNN engines following data augmentation. Notably, Resnet50 emerged as the leading model. The models achieved a ROC-AUC of 0.962 for Resnet50, 0.956 for VGG16, 0.931 for EfficientNetV2, 0.929 for InceptionV3, and 0.915 for Densenet169.

TABLE 8
Average evaluation metrics for the trained models on the
augmented binary dataset (sorted based on performance).

| | | Training | Validation | Test Set Metrics | | | |
|---|---|---|---|---|---|---|---|
| Dataset | Model | Accuracy | Accuracy | Accuracy | Precision | Recall | F1-score |
| Binary | Resnet50 | 0.952 | 0.933 | 0.877 | 0.941 | 0.801 | 0.865 |
| augmented | VGG16 | 0.877 | 0.897 | 0.832 | 0.965 | 0.688 | 0.802 |
| | InceptionV3 | 0.920 | 0.893 | 0.831 | 0.927 | 0.715 | 0.807 |
| | EfficientNetV2 | 0.885 | 0.891 | 0.825 | 0.975 | 0.666 | 0.791 |
| | DenseNet169 | 0.946 | 0.927 | 0.771 | 0.952 | 0.570 | 0.713 |

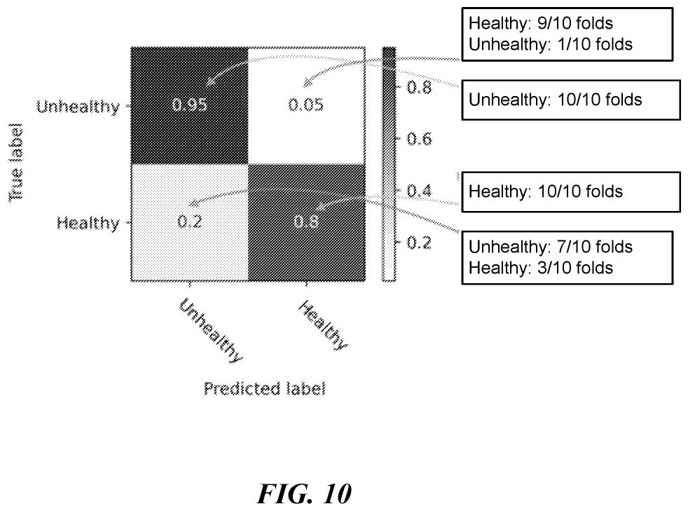

Examining the performance of the optimal model, specifically the Resnet50 with the augmented dataset, a detailed analysis of the metrics per class for this CNN becomes possible. Table 9 displays the outcomes of a 10-fold cross-validation process, highlighting the model's overall consistent performance throughout the iterations. Additionally, FIG. 10 illustrates the aggregated confusion matrix for the Resnet50 model. This matrix synthesizes the predictions from all ten folds applied to the augmented dataset. The aggregation process involved calculating the median predicted class for each instance over the multiple folds, thereby creating a unified prediction that reflects the consensus of the model's behavior across the test set.

TABLE 9
Results of 10-fold Cross-Validation for the augmented
binary dataset on Resnet50.

| Iteration of 10- | | | | F1- |
|---|---|---|---|---|
| Fold | Accuracy | Precision | Recall | score |
| Iteration 1 | 0.769 | 0.948 | 0.557 | 0.702 |
| Iteration 2 | 0.761 | 0.960 | 0.531 | 0.684 |
| Iteration 3 | 0.791 | 0.951 | 0.601 | 0.737 |
| Iteration 4 | 0.782 | 0.970 | 0.570 | 0.718 |
| Iteration 5 | 0.782 | 0.932 | 0.596 | 0.727 |
| Iteration 6 | 0.778 | 0.943 | 0.579 | 0.717 |
| Iteration 7 | 0.793 | 0.928 | 0.623 | 0.745 |
| Iteration 8 | 0.767 | 0.961 | 0.544 | 0.695 |
| Iteration 9 | 0.778 | 0.963 | 0.566 | 0.713 |
| Iteration 10 | 0.771 | 0.969 | 0.548 | 0.700 |

In this example and out of the 468 images in the testing set, the model successfully classified 411 images. The breakdown of correctly classified images by category is as follows: unhealthy (N=228, Accuracy=95%) and healthy (N=183, Accuracy=80%). The incorrectly classified images exhibited redness (in instances of engorgement mislabeled as unhealthy, N=26), and incomplete images (where the nipple and breast were not fully visible, N=12). Table 10 provides a detailed summary of the CNN engine's performance per class on the augmented test set.

TABLE 10
Summary of the detection results per class: accuracy,
precision, recall, F1-score and support (number of
samples per class) using the Resnet50 architecture.

| | | | | F1- | |
|---|---|---|---|---|---|
| Class | Accuracy | Precision | Recall | score | Support |
| Unhealthy | 0.950 | 0.835 | 0.950 | 0.889 | 240 |
| Healthy | 0.802 | 0.938 | 0.802 | 0.865 | 228 |

### Image Quality, Image Interpretations, and Video Interpretations

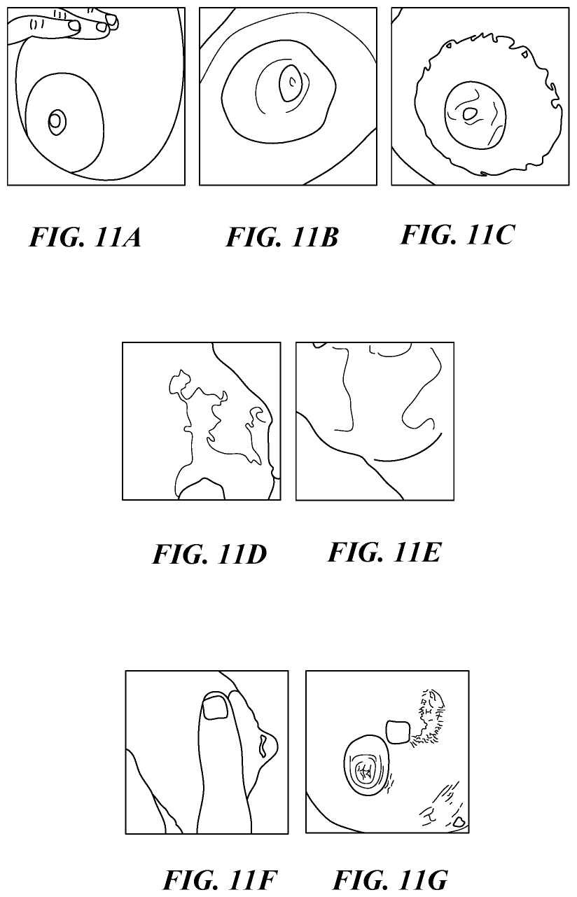

During the evaluation of misclassification incidents within the image dataset study, it was observed that numerous image quality issues significantly influenced the deep learning model's diminished performance. As illustrated in FIGS. 11A, 11B, and 11C, high-quality images that are suitable for testing on the system are depicted. These line drawing images either completely or nearly completely display the nipple from an appropriate distance, facilitating accurate diagnosis without incorporating extraneous background details that could lead to misinterpretation by the model. In contrast, the primary deficiency observed in FIGS. 11D and 11E is the absence of the nipple, which challenges the model's ability to correctly associate the images with typical breast figures. Despite visible indications of conditions such as mastitis or engorgement, the incompleteness of these images impairs accurate model assessment. In examples shown in FIGS. 11F and 11G, the accuracy of the model is further compromised by the presence of hands or fingers, nail polish, and obstructions caused by extraneous objects. These elements obscure critical areas of interest and introduce visual elements not accounted for in the model's training dataset, thereby adversely affecting the model's interpretative accuracy.

There were other issues noted in the preprocessing phase that once fixed, can improve the deep learning model's performance. For example, having the image of both breasts instead of one affects prediction, especially if one breast will have a different condition in relation to the other. If the image shows some background components or partially the baby's face for images taken from lactation web examples, there are detection issues that were corrected by cropping the image to the area of interest. Images exhibiting low resolution are also subject to performance degradation, particularly if they are initially smaller than the dimensions stipulated by the data augmentation algorithm and are subsequently enlarged. Notably, several images that fall into this category and experienced misclassification were resized appropriately, following which the model successfully classified them correctly. This observation underscores the critical influence of image resolution and proper preprocessing on the accuracy of model performance.

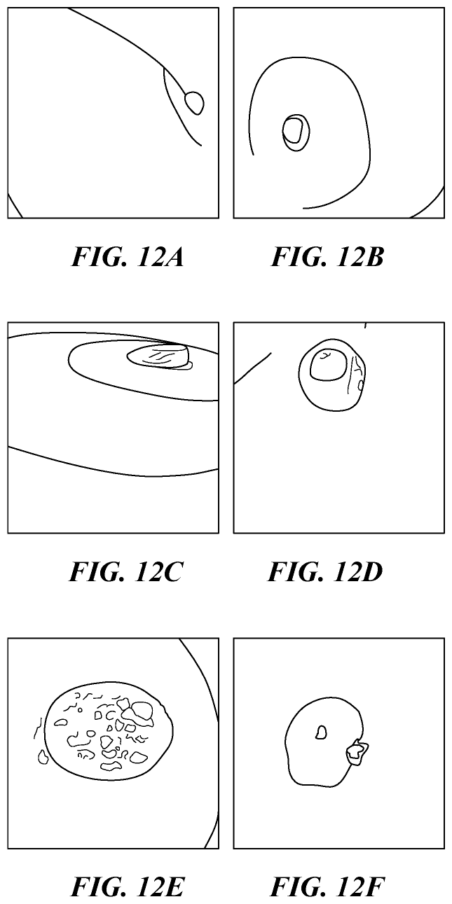

In the field of machine learning and, more specifically, in the application of CNNs for image classification tasks, the accuracy and reliability of the model's predictions are important. CNNs have been widely adopted due to their ability to automatically and adaptively learn spatial hierarchies of features from input images. However, despite their robustness and efficacy, CNN engines are not immune to misclassification errors. Identifying and understanding the underlying causes of these errors is crucial for improving model performance and ensuring the reliability of its applications, particularly in critical domains such as medical imaging. FIGS. 12A to 12F show line drawing images leading to correct prediction from several classes of model misclassification, including: 1) common visual features in the images between the classes; 2) wrong positioning of the breast in the image; 3) a lack of variety of images belonging to specific cases in the dataset; and 4) presence of an extraneous object in the frame.

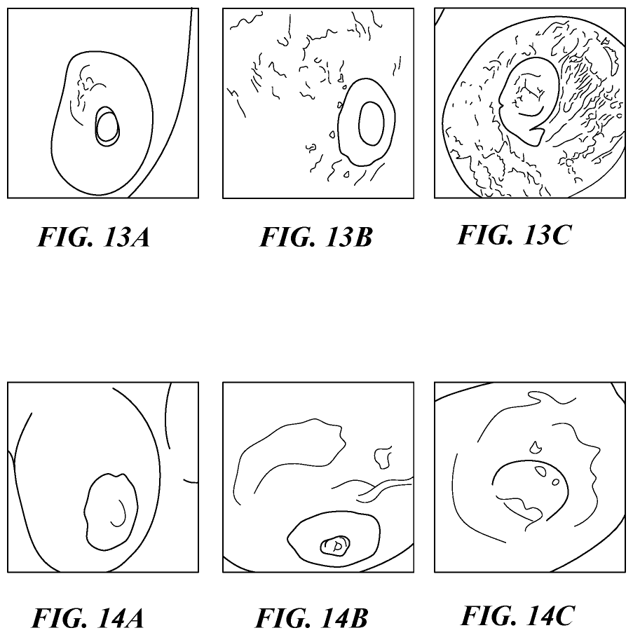

In this example, the deep learning model adopted for the present technology may be encountered challenges in distinguishing between classes that share common visual features. These similarities in the images can lead to confusion for the deep learning model, causing it to incorrectly assign a class label to an image. This is particularly problematic when the shared features are prominent or common across multiple classes. An additional source of confusion for the model arises from nipples bearing scars from previous medical interventions, such as breast biopsies. The model erroneously classifies these as damaged nipples, despite the breast being healthy at the time of imaging, as demonstrated in FIG. 13A. In contrast, FIGS. 13B and 13C are categorized respectively under nipple damage and mastitis. Specifically, FIG. 13B illustrates a case of mastitis characterized by extensive redness across the breast and skin peeling. The minimal variation in skin tone complicates the model's ability to distinguish this condition from nipple dermatitis, which presents similar dermatological features. In FIG. 13C, the misclassification likely stems from the absence of visible nipple damage, while the dermatitis, primarily located on the areola, could be misinterpreted as discoloration of the areola rather than a distinct condition. This illustrates the model's challenges in accurately identifying conditions when presented with subtle or overlapping symptoms.

The dataset's limitations in representing the full spectrum of specific cases have been observed to hinder the deep learning model's ability to generalize its learning effectively. A lack of variety in the images for certain classes can cause the model to overfit to the limited examples it has been trained on, thereby reducing its accuracy when encountering new, unseen images. A principal factor contributing to model confusion between healthy breasts and those with mastitis includes images depicting breast engorgement and early-stage mastitis, which can appear similar, particularly in terms of swollen breast shape and mild redness, as shown in FIGS. 14A and 14B. The scarcity of sufficient images of breast engorgement for creating a separate classification, coupled with the fact that while engorgement itself is not inherently problematic, it can progress to mastitis if untreated, has led to numerous instances where the model erroneously classified engorged breasts as mastitis.

Furthermore, FIG. 14C displays a breast affected by Hand-foot-and-mouth disease, characterized by blisters on the nipple and redness. This redness led the deep learning model to misclassify the condition as mastitis. Such misclassifications, involving distinct but visually overlapping features, can also pose classification challenges for human experts, especially given the rarity of some conditions. The limited availability of diverse images publicly accessible, encompassing various skin tones, breast shapes, and nipple sizes, exacerbates these challenges. It is posited that the acquisition of a broader array of images featuring rare disorders and the implementation of enhanced data augmentation techniques could significantly improve the model's classification accuracy.

In this disclosure, another example of misclassification includes conditions that occur together, such as a breast abscess concentrated behind the nipple accompanied by signs of nipple damage. This instance was among the rare occurrences of simultaneous conditions in the dataset and underscored the reality that lactation consultants encounter patients with similar cases. This brings to light the necessity for systems that either recognize multiple conditions or decide which condition is the most severe to prioritize patient care. A case of granulomatous mastitis, often misclassified as nipple damage due to the presence of nipple scarring, also highlights the rarity of such specific cases in the dataset.

Furthermore, the dataset includes images that show breasts in conditions of engorgement and nipple damage. In one example, an engorged breast with an inverted nipple appears lighter in the center, leading to a misclassification as a nipple bleb. In another example, the proximity and nature of nipple damage near a blood blister cause a reflection that suggests the presence of a nipple bleb, resulting in another misclassification. These images with distinct features present classification challenges, even for humans, primarily because some conditions are infrequently encountered. The variety of cases across different skin tones, breast shapes, and nipple sizes is limited in publicly available images, suggesting that incorporating more images of rare disorders and expanding data augmentation techniques could significantly enhance model classification. Additionally, issues with image angle and proximity, such as a picture taken too close to the breast, increase the likelihood of misclassification.

In the present disclosure, the significance of conducting video evaluations lies in the creation of a highly naturalistic dataset that captures how parents utilize smartphones to record videos of breast conditions without any guidance. This process enables a thorough examination of potential, unforeseen image quality issues that might arise. Understanding these issues provides crucial insights for future design enhancements of user interfaces, specifically tailored for optimal use of such computer vision systems by untrained users. Further, this approach ensures that the data evaluation system is accessible and effective for everyday applications, thereby increasing its utility and user-friendliness.

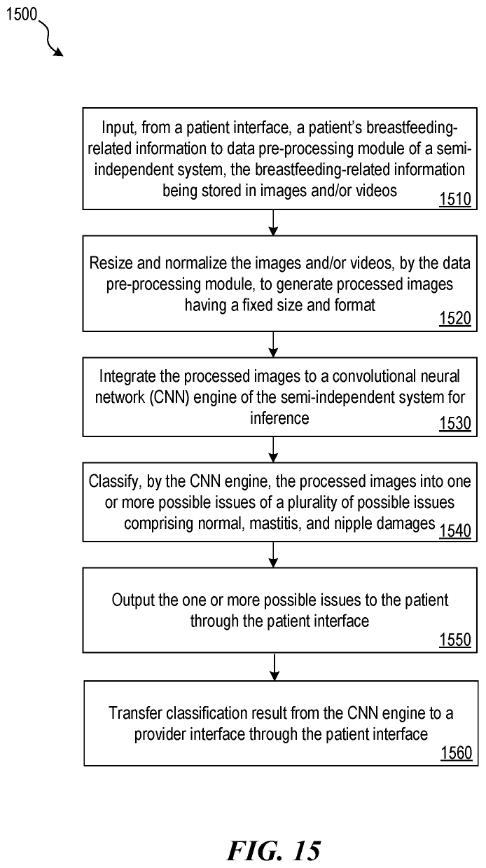

FIG. 15 is a flow chart illustrating a method 1500 for breastfeeding related issue management in accordance with various embodiments of the present technology. The method 1500 includes inputting, from a patient interface, a patient's breastfeeding-related information to data pre-processing module of a semi-independent system, at 1510. For example, patient information, symptoms and condition images can be captured at the patient interface 104, as shown in FIG. 1.

In addition, the method 1500 includes resizing and normalizing the images and/or videos, by the data pre-processing module, to generate processed images having a fixed size and format, at 1520. For example, the image resizing and normalization module 214 can be utilized in the AI-mediated patient management pipeline 210 to generate processed images having fixed size and format of 224×224 pixels with RGB channels, as shown in FIG. 2.

The method 1500 also includes integrating the processed images to a convolutional neural network (CNN) engine of the semi-independent system for inference, at 1530. For example, the processed images can be forwarded from the image resizing and normalization module 214 to the subsequent deep learning engine 216 for classification, as shown in FIG. 2.

Moreover, the method 1500 includes classifying, by the CNN engine, the processed images into one or more possible issues of a plurality of possible issues comprising normal, mastitis, and nipple damages, at 1540. For example, one or more possible issues can be identified based on the possibilities of classification results generated by the deep learning engine 216. In some other examples, the categories of the plurality of possible issues include breast cancer, fibrocystic breasts, breast cysts, papilloma, gynecomastia, breast abscess, breast calcification, and/or breast atrophy.

Further, the method 1500 includes outputting the one or more possible issues to the patient through the patient interface, at 1550. For example, the possible issues can be forwarded from the deep learning engine 216 of the semi-independent system 106 back to the patient 102, as shown in FIG. 1. Alternatively, possible issues can be manually reviewed by the LC 124 through the provider interface 122.

Lastly, the method 1500 includes transferring classification result from the CNN engine to a provider interface through the patient interface, at 1560. For example, classification results including high possibility possible issues and/or processed images can be forwarded from the CNN engine 216 to the provider interface 122, through the patient interface 104, as shown in FIG. 1.

In some examples, the present technology can deploy a user interface and educational resources to aid parents in capturing images for use with the AI mediated triaging system. These systems are designed to provide straightforward guidelines on framing the breast to avoid occlusion, advising against using fingers to point out areas of interest, and ensuring the camera captures the entire breast, including the nipple, areola, and breast tissue. These guidelines are presented in an easy-to-understand format, while more advanced systems may offer immediate feedback on image quality.

In some examples, the present technology can utilize non-RGB images to triage breastfeeding-related conditions. Additionally, it can integrate patient-reported data concerning the onset, location, symptoms, and intensity of pain, which are crucial for diagnosing and addressing issues related to breastfeeding pain. The present technology also supports fully developed hybrid-remote consultations, where patients provide initial input through a questionnaire, and subsequent image sharing between the patient and provider facilitates a detailed visualization of the condition's severity before administering care. This integration of visual and symptomatic information in remote consultations enhances the diagnostic process and allows LCs to provide customized, timely care.

In some examples, the present technology offers a holistic approach to determining when to flag a patient as at-risk within the AI-mediated lactation care system. It merges quantitative data from image detection and pain assessment with clinical insights. Applications of this technology include providing actionable feedback for managing breastfeeding pain, reliably identifying urgent cases, and facilitating prompt interventions to improve outcomes in lactation care.

The present disclosure demonstrates the feasibility of AI-mediated triaging of breast conditions for lactating women. A first step of the present technology in this domain is using RGB breast images to triage healthy from unhealthy breasts in mastitis spectrum disease and nipple damage spectrum (caused by poor breastfeeding techniques, milk supply, and skin conditions). A deep learning model is further incorporated to classify three breast conditions across images. The evaluation of this deep learning model based on the dataset demonstrates the feasibility of such an application.

The CNN engine described in the present disclosure can be integrated into a telehealth pipeline for postpartum lactation care, helping LCs classify and organize patients based on the severity of their condition or the level of certainty that they are experiencing health issues. Additionally, the image evaluation system of the present technology can help to track patient disease progression and aid newly qualified LCs by providing faster decision-making support. Furthermore, automation of patient responses can be achieved by categorizing issues based on their classification, streamlining the care process, and ensuring prompt patient attention. The present technology allows a more comprehensive understanding of breast health for postpartum mothers and empowers them to take proactive steps in maintaining their well-being.

In a first example, the present disclosure describes an AI mediated patient management system, comprising: a patient interface configured to collect breastfeeding-related information from a patient, the breastfeeding-related information being stored in images and/or videos; a semi-independent system connected to the patient interface and configured to propose actionable feedback to the patient, the semi-independent system comprising: a data pre-processing module connected with the patient interface and configured to resize and normalize the images and/or videos forwarded by the patient interface, wherein the data pre-processing module generates processed images having a fixed size and format, and a CNN engine having a final layer and a softmax activation function that classify the processed images into one or more possible issues of a plurality of possible issues, comprising normal, mastitis, and nipple damage; a patient triage module that includes a data base containing the one or more possible issues; and a provider interface connected to the patient triage module and a lactation consultant, the provider interface configured to enable the lactation consultant to communicate with the patient to diagnose one of the plurality of possible issues, wherein the patient interface is directly connected to the provider interface and the semi-independent system is not directly connected to the patient trigae system, and wherein the classification of the processed images is transferred from the semi-independent system to the provider interface through the patient interface.

In a second example, the present disclosure describes the patient management system of the first example, wherein the images use a RGB color model, and the videos include pixels having associated RGB values.

In a third example, the present disclosure describes the patient management system of the first example, wherein the breastfeeding-related information comprises symptoms, pain levels, location, and duration.

In a fourth example, the present disclosure describes the patient management system of the first example, wherein the processed images are presented in a square geometry.

In a fifth example, the present disclosure describes the patient management system of the first example, wherein the processed images have a fixed size and format of 224×224 pixels with RGB channels.

In a sixth example, the present disclosure describes the patient management system of the first example, wherein the CNN engine is a deep learning engine consists of at least one of a Resnet50 engine, a VGG16 engine, an EfficientNetV2 engine, an InceptionV3 engine, and a DenseNet169 engine.

In a seventh example, the present disclosure describes the patient management system of the first example, wherein the plurality of categories further comprises breast cancer, fibrocystic breasts, breast cysts, papilloma, gynecomastia, breast abscess, breast calcification, and/or breast atrophy.

In an eighth example, the present disclosure describes the patient management system of the first example, wherein the videos are short scenes displaying breast area of the patient that represent various motions, angles and distances from the patient.

In a ninth example, the present disclosure describes the patient management system of the eighth example, wherein the semi-independent system is configured to extract frames from the videos, and resize and normalize the extracted frames.

In a tenth example, the present disclosure describes the patient management system of the first example, wherein the CNN engine comprises a convolutional neural network VGG16, with a built-in pre-trained model using PyTorch library for image classification.

In an eleventh example, the present disclosure describes a method for breastfeeding related issue management, comprising: inputting, from a patient interface, a patient's breastfeeding-related information to data pre-processing module of a semi-independent system, the breastfeeding-related information being stored in images and/or videos; resizing and normalizing the images and/or videos, by the data pre-processing module, to generate processed images having a fixed size and format; integrating the processed images to a CNN engine of the semi-independent system for inference; classifying, by the CNN engine, the processed images into one or more possible issues of a plurality of possible issues comprising normal, mastitis, and nipple damages; outputting the one or more possible issues to the patient through the patient interface; and transferring classification result from the CNN engine to a provider interface through the patient interface, wherein the patient interface is directly connected to the provider interface and the semi-independent system is not directly connected to the patient trigae system.

In a twelfth example, the present disclosure describes the method of the eleventh example, further comprising proposing actionable feedback to the patient based on the one or more possible issues.

In a thirteenth example, the present disclosure describes the method of the eleventh example, further comprising manually reviewing, by a lactation consultant connected to the provider interface, the classification result and the plurality of possible issues.

In a fourteenth example, the present disclosure describes the method of the thirteenth example, further comprising communicating, by the lactation consultant, with the patient to diagnose in accordance with the one or more possible issues.

In a fifteenth example, the present disclosure describes the method of the eleventh example, further comprising framing the videos by the data pre-processing module.

In a sixteenth example, the present disclosure describes the method of the fifteenth example, wherein the resizing of the images and/or videos includes chopping the images and/or video frames into square shape processed image without losing its original resolution.

In a seventeenth example, the present disclosure describes the method of the sixteenth example, wherein the normalizing of the images and/or videos includes modifying pixel values in the images and/or video frames to have a specific statistical distribution.

In an eighteenth example, the present disclosure describes the method of the eleventh example, wherein the processed images have fixed size and format of 224×224 pixels with RGB channels.

In a nineteenth example, the present disclosure describes the method of the eleventh example, wherein the plurality of categories further comprises breast cancer, fibrocystic breasts, breast cysts, papilloma, gynecomastia, breast abscess, breast calcification, and/or breast atrophy.

In a twentieth example, the present disclosure describes a system comprising one or more computers and one or more storage devices storing instructions that are operable, when executed by the one or more computers, to cause the one or more computers to perform operations comprising: inputting, from a patient interface, a patient's breastfeeding-related information to data pre-processing module of a semi-independent system, the breastfeeding-related information being stored in images and/or videos; resizing and normalizing the images and/or videos, by the data pre-processing module, to generate processed images having a fixed size and format; integrating the processed images to a CNN engine of the semi-independent system for inference; classifying, by the CNN engine, the processed images into one or more possible issues of a plurality of possible issues comprising normal, mastitis, and nipple damages; outputting the one or more possible issues to the patient through the patient interface; and transferring classification result from the CNN engine to a provider interface through the patient interface, wherein the patient interface is directly connected to the provider interface and the semi-independent system is not directly connected to the patient trigae system.

It is understood that the various disclosed embodiments may be implemented individually, or collectively, using devices comprised of various electrical components, electronics hardware and/or software modules and components. These devices, for example, may comprise a processor, a memory unit, an interface that are communicatively connected to each other, and may range from desktop and/or laptop computers, to mobile devices and the like. The processor and/or controller can perform various disclosed operations based on execution of program code that is stored on a storage medium. The processor and/or controller can, for example, be in communication with at least one memory and with at least one communication unit that enables the exchange of data and information, directly or indirectly, through the communication link with other entities, devices and networks. The communication unit may provide wired and/or wireless communication capabilities in accordance with one or more communication protocols, and therefore it may comprise the proper transmitter/receiver antennas, circuitry and ports, as well as the encoding/decoding capabilities that may be necessary for proper transmission and/or reception of data and other information.

Various information and data processing operations described herein may be implemented in one embodiment by a computer program product, embodied in a computer-readable medium, including computer-executable instructions, such as program code, executed by computers in networked environments. A computer-readable medium may include removable and non-removable storage devices including, but not limited to, Read Only Memory (ROM), Random Access Memory (RAM), compact discs (CDs), digital versatile discs (DVD), etc. Therefore, the computer-readable media that is described in the present application comprises non-transitory storage media. Generally, program modules may include routines, programs, objects, components, data structures, etc. that perform particular tasks or implement particular abstract data types. Computer-executable instructions, associated data structures, and program modules represent examples of program code for executing steps of the methods disclosed herein. The particular sequence of such executable instructions or associated data structures represents examples of corresponding acts for implementing the functions described in such steps or processes.

The foregoing description of embodiments has been presented for purposes of illustration and description. The foregoing description is not intended to be exhaustive or to limit embodiments of the present invention to the precise form disclosed, and modifications and variations are possible in light of the above teachings or may be acquired from practice of various embodiments. The embodiments discussed herein were chosen and described in order to explain the principles and the nature of various embodiments and its practical application to enable one skilled in the art to utilize the present invention in various embodiments and with various modifications as are suited to the particular use contemplated. While operations are depicted in the drawings in a particular order, this should not be understood as requiring that such operations be performed in the particular order shown or in sequential order, or that all illustrated operations be performed, to achieve desirable results. The features of the embodiments described herein may be combined in all possible combinations of methods, apparatus, modules, and systems.

Only a few implementations and examples are described and other implementations, enhancements and variations can be made based on what is described and illustrated in this patent document.

What is claimed is:

1. An artificial intelligent (AI) mediated patient management system, comprising:
a patient interface configured to collect breastfeeding-related information from a patient, the breastfeeding-related information being stored in images and/or videos;
a semi-independent system connected to the patient interface and configured to propose actionable feedback to the patient, the semi-independent system comprising:
a data pre-processing module connected with the patient interface and configured to resize and normalize the images and/or videos forwarded by the patient interface, wherein the data pre-processing module generates processed images having a fixed size and format, and
a convolutional neural network (CNN) engine having a final layer and a softmax activation function that classify the processed images into one or more possible issues of a plurality of possible issues, comprising normal, mastitis, and nipple damage;
a patient triage module that includes a data base containing the one or more possible issues; and
a provider interface connected to the patient triage module and a lactation consultant, the provider interface configured to enable the lactation consultant to communicate with the patient to diagnose one of the plurality of possible issues,
wherein the patient interface is directly connected to the provider interface and the semi-independent system is not directly connected to the patient trigae system, and
wherein the classification of the processed images is transferred from the semi-independent system to the provider interface through the patient interface.

2. The patient management system of claim 1, wherein the images use a RGB color model, and the videos include pixels having associated RGB values.

3. The patient management system of claim 1, wherein the breastfeeding-related information comprises symptoms, pain levels, location, and duration.

4. The patient management system of claim 1, wherein the processed images are presented in a square geometry.

5. The patient management system of claim 1, wherein the processed images have a fixed size and format of 224×224 pixels with RGB channels.

6. The patient management system of claim 1, wherein the CNN engine is a deep learning engine consists of at least one of a Resnet50 engine, a VGG16 engine, an EfficientNetV2 engine, an InceptionV3 engine, and a DenseNet169 engine.

7. The patient management system of claim 1, wherein the plurality of categories further comprises breast cancer, fibrocystic breasts, breast cysts, papilloma, gynecomastia, breast abscess, breast calcification, and/or breast atrophy.

8. The patient management system of claim 1, wherein the videos are short scenes displaying breast area of the patient that represent various motions, angles and distances from the patient.

9. The patient management system of claim 8, wherein the semi-independent system is configured to extract frames from the videos, and resize and normalize the extracted frames.

10. The patient management system of claim 1, wherein the CNN engine comprises a convolutional neural network VGG16, with a built-in pre-trained model using PyTorch library for image classification.

11. A method for breastfeeding related issue management, comprising:
inputting, from a patient interface, a patient's breastfeeding-related information to data pre-processing module of a semi-independent system, the breastfeeding-related information being stored in images and/or videos;
resizing and normalizing the images and/or videos, by the data pre-processing module, to generate processed images having a fixed size and format;
integrating the processed images to a convolutional neural network (CNN) engine of the semi-independent system for inference;
classifying, by the CNN engine, the processed images into one or more possible issues of a plurality of possible issues comprising normal, mastitis, and nipple damages;
outputting the one or more possible issues to the patient through the patient interface; and
transferring classification result from the CNN engine to a provider interface through the patient interface,
wherein the patient interface is directly connected to the provider interface and the semi-independent system is not directly connected to the patient trigae system.

12. The method of claim 11, further comprising proposing actionable feedback to the patient based on the one or more possible issues.

13. The method of claim 11, further comprising manually reviewing, by a lactation consultant connected to the provider interface, the classification result and the plurality of possible issues.

14. The method of claim 13, further comprising communicating, by the lactation consultant, with the patient to diagnose in accordance with the one or more possible issues.

15. The method of claim 11, further comprising framing the videos by the data pre-processing module.

16. The method of claim 15, wherein the resizing of the images and/or videos includes chopping the images and/or video frames into square shape processed image without losing its original resolution.

17. The method of claim 16, wherein the normalizing of the images and/or videos includes modifying pixel values in the images and/or video frames to have a specific statistical distribution.

18. The method of claim 11, wherein the processed images have fixed size and format of 224×224 pixels with RGB channels.

19. The method of claim 11, wherein the plurality of categories further comprises breast cancer, fibrocystic breasts, breast cysts, papilloma, gynecomastia, breast abscess, breast calcification, and/or breast atrophy.

20. A system comprising one or more computers and one or more storage devices storing instructions that are operable, when executed by the one or more computers, to cause the one or more computers to perform operations comprising:
inputting, from a patient interface, a patient's breastfeeding-related information to data pre-processing module of a semi-independent system, the breastfeeding-related information being stored in images and/or videos;
resizing and normalizing the images and/or videos, by the data pre-processing module, to generate processed images having a fixed size and format;
integrating the processed images to a convolutional neural network (CNN) engine of the semi-independent system for inference;
classifying, by the CNN engine, the processed images into one or more possible issues of a plurality of possible issues comprising normal, mastitis, and nipple damages;
outputting the one or more possible issues to the patient through the patient interface; and
transferring classification result from the CNN engine to a provider interface through the patient interface,
wherein the patient interface is directly connected to the provider interface and the semi-independent system is not directly connected to the patient trigae system.
